(19) United States
(12) Patent Application Publication
(10) Pub. No.: US 2021/0054076 A1
(43) Pub. Date: Feb. 25, 2021

(54) CYTOTOXICITY-INDUCING THERAPEUTIC AGENT

(71) Applicant: Chugai Seiyaku Kabushiki Kaisha, Tokyo (JP)

(72) Inventor: Naoki KIMURA, Kanagawa (JP)

(21) Appl. No.: 16/959,288

(22) PCT Filed: Jan. 4, 2019
(86) PCT No.: PCT/JP2019/000008

§ 371 Date: Jun. 30, 2020

(30) Foreign Application Priority Data
Jan. 5, 2018 (JP) 2018-000701

(51) Int. Cl. C07K 16/28; A61P 35/00

(52) U.S. Cl. CPC C07K 16/2809; A61P 35/00

(57) ABSTRACT

The present inventors discovered novel multispecific antigen-binding molecules with excellent cellular cytotoxicity and high stability, which comprise a first domain comprising a first antibody variable region that binds to Claudin 6, a second domain comprising a second antibody variable region that binds to T cell receptor complex. Since the molecules of the present invention show a strong cytotoxicity against cells and tissues expressing Claudin 6, it is possible to produce novel pharmaceutical compositions comprising the multispecific antigen-binding molecules for treating or preventing various cancers.

### TECHNICAL FIELD

The present invention relates to multispecific antigen-binding molecules that comprise a first domain comprising a first antibody variable region which binds to human Claudin 6 (CLDN6), a second domain comprising a second antibody variable region which binds to T cell receptor complex and a third domain comprising an Fc region, uses thereof, and such.

### BACKGROUND ART

Cancer is one of the leading causes of death worldwide. With the exception of certain carcinomas, tumors are often inoperable when they are found. Conventional cancer treatments include radiation therapy, chemotherapy, and immunotherapy. These treatments are often not effective enough and eventually cancer recurrence or metastasis occurs after the treatment. Lack of tumor specificity is one of the factors that limit the maximum efficacy; therefore, more tumor-specific molecular targeted therapy has become an additional viable option in cancer treatment.

Antibodies are drawing attention as pharmaceuticals since they are highly stable in plasma and have few side effects. Among multiple therapeutic antibodies, some types of antibodies require effector cells to exert an anti-tumor response. Antibody dependent cell-mediated cytotoxicity (ADCC) is a cytotoxicity exhibited by effector cells against antibody-bound cells via binding of the Fc region of the antibody to Fc receptors present on NK cells and macrophages. To date, multiple therapeutic antibodies that can induce ADCC to exert anti-tumor efficacy have been developed as pharmaceuticals for treating cancer (NPL 1). Therapies targeting tumor-specific expressed antigens using conventional therapeutic antibodies show excellent anti-tumor activities, while administration of such antibodies could not always lead to satisfactory outcomes.

In addition to the antibodies that adopt ADCC by recruiting NK cells or macrophages as effector cells, T cell-recruiting antibodies (TR antibodies) that adopt cytotoxicity by recruiting T cells as effector cells have been known since the 1980s (NPL 2 to 4). A TR antibody is a bispecific antibody that recognizes and binds to any one of the subunits forming a T-cell receptor complex on T-cells, in particular the CD3 epsilon chain, and an antigen on cancer cells. Several TR antibodies are currently being developed. Catumaxomab, which is a TR antibody against EpCAM, has been approved in the EU for the treatment of malignant ascites. Furthermore, a type of TR antibody called “bispecific T-cell engager (BiTE)” has been recently found to exhibit a strong anti-tumor activity (NPL 5 and 6). Blinatumomab, which is a BiTE molecule against CD19, received FDA approval first in 2014. Blinatumomab has been proved to exhibit a much stronger cytotoxic activity against CD19/CD20-positive cancer cells in vitro compared with Rituximab, which induces antibody-dependent cell-mediated cytotoxicity (ADCC) and complement-dependent cytotoxicity (CDC) (NPL 7).

However, it is known that a trifunctional antibody binds to both a T-cell and a cell such as an NK cell or macrophage at the same time in a cancer antigen-independent manner, and as a result receptors expressed on the cells are cross-linked, and expression of various cytokines is induced in a cancer antigen-independent manner. Systemic administration of a trifunctional antibody is thought to cause cytokine storm-like side effects as a result of such induction of cytokine expression. In fact, it has been reported that, in the phase I clinical trial, a very low dose of 5 micro g/body was the maximum tolerance dose for systemic administration of catumaxomab to patients with non-small cell lung cancer, and that administration of a higher dose causes various severe side effects (NPL 8). When administered at such a low dose, catumaxomab can never reach the effective blood level. That is, the expected anti-tumor effect cannot be achieved by administrating catumaxomab at such a low dose.

Meanwhile, unlike catumaxomab, BiTE has no Fc gamma receptor-binding site, and therefore it does not cross-link the receptors expressed on T-cells and cells such as NK cells and macrophages in a cancer antigen-independent manner. Thus, it has been demonstrated that BiTE does not cause cancer antigen-independent cytokine induction which is observed when catumaxomab is administered. However, since BiTE is a modified low-molecular-weight antibody molecule without an Fc region, the problem is that its blood half-life after administration to a patient is significantly shorter than IgG-type antibodies conventionally used as therapeutic antibodies. In fact, the blood half-life of BiTE administered in vivo has been reported to be about several hours (NPL 9 and 10). In the clinical trials of blinatumomab, it is administered by continuous intravenous infusion using a minipump. This administration method is not only extremely inconvenient for patients but also has the potential risk of medical accidents due to device malfunction or the like. Thus, it cannot be said that such an administration method is desirable.

Claudin family is the family of cell membrane proteins of approximately 23 kD in molecular weight which have four transmembrane domains and constitute tight junctions. The Claudin family includes 24 members in humans and mice, and each member of the Claudins is known to exhibit a very unique expression pattern depending on each epithelial cell type (NPL 11 to 14). In the sheet of epithelial cells, a mechanism works to prevent substances from leaking (diffusing) in the intercellular spaces, and cell-cell adhesion systems called tight junctions have been shown to really play a central role as a “barrier” in the mechanism to prevent leakage.

Regarding anti-CLDN6 antibodies, monospecific antibodies against CLDN6 has been reported to have ADCC activity or internalization activity against CLDN6 positive cancer lines (PTL 1 to 5). Bispecific molecule targeting CLDN6 was made in the molecule format of BiTE (PTL 6 to 7), however, as mentioned above, BiTE is a modified low-molecular-weight antibody molecule without an Fc region and show significantly shorter blood half-life compared with IgG-type antibodies, which would cause inconvenience for their use as a therapeutic drug.

### CITATION LIST

### Patent Literature

[PTL 1] WO2009/087978
[PTL 2] WO2011/057788
[PTL 3] WO2012/003956
[PTL 4] WO2012/156018
[PTL 5] WO2015/069794
[PTL 6] WO2014/075697
[PTL 7] WO2014/075788

### Non-Patent Literature

[NPL 1] Clin Cancer Res. 2010 Jan. 1; 16(1):11-20.
[NPL 2] Nature. 1985 Apr. 18-24; 314(6012):628-31.
[NPL 3] Int J Cancer. 1988 Apr. 15; 41(4):609-15.
[NPL 4] Proc Natl Acad Sci USA. 1986 March; 83(5):1453-7.
[NPL 5] Proc Natl Acad Sci USA. 1995 Jul. 18; 92(15):7021-5.
[NPL 6] Drug Discov Today. 2005 Sep. 15; 10(18):1237-44.
[NPL 7] Int J Cancer. 2002 Aug. 20; 100(6):690-7.
[NPL 8] Cancer Immunol Immunother (2007) 56 (10), 1637-44
[NPL 9] Cancer Immunol Immunother. (2006) 55 (5), 503-14
[NPL 10] Cancer Immunol Immunother. (2009) 58 (1), 95-109
[NPL 11] Furuse and Tsukita, TRENDS in Cell Biology 2006, 16: 181
[NPL 12] Wilcox, et al., Cell 2001, 104: 165
[NPL 13] Rahner, et al., GASTROENTEROLOGY 2001, 120: 411
[NPL 14] Morita, et al., Proc. Natl. Acad. Sci. USA 1999, 96: 511

### SUMMARY OF INVENTION

### Technical Problem

An objective of the present invention is to provide multispecific antigen-binding molecules that enable cancer treatment by having T cells close to CLDN6-expressing cells and using the cytotoxicity of T cells against CLDN6-expressing cancer cells, methods for producing the multispecific antigen-binding molecules, and therapeutic agents comprising such a multispecific antigen-binding molecule as an active ingredient for inducing cellular cytotoxicity. Another objective of the present invention is to provide pharmaceutical compositions for use in treating or preventing various cancers, which comprise one of the above-mentioned antigen-binding molecules as an active ingredient, and therapeutic methods using the pharmaceutical compositions.

### Solution to Problem

The inventors found that multispecific antigen-binding molecules that comprise a first domain comprising a first antibody variable region which binds to human CLDN6, and a second domain comprising a second antibody variable region which binds to T-cell receptor complex can damage cells expressing CLDN6, and exert a superior cytotoxic/antitumor activity. The present invention provides the multispecific antigen-binding molecules and pharmaceutical compositions that can treat various cancers, especially those associated with CLDN6 such as CLDN6-positive tumors, by comprising the antigen-binding molecule as an active ingredient.

More specifically, the present invention provides the following:
[1] A multispecific antigen-binding molecule that comprises:
   (1) a first domain comprising a first antigen-binding domain which binds to human CLDN6,
   (2) a second domain comprising a second antigen-binding domain which binds to T-cell receptor complex, and
   (3) a third domain comprising an Fc region.
[2] The multispecific antigen-binding molecule of [1], wherein the multispecific antigen-binding molecule has cytotoxic activity.
[3] The multispecific antigen-binding molecule of [2], wherein the cytotoxic activity is T-cell-dependent cytotoxic activity.
[4] The multispecific antigen-binding molecule of any one of [1] to [3], wherein the first antigen-binding domain of (1) specifically binds to human CLDN6.
[5] The multispecific antigen-binding molecule of any one of [1] to [4], wherein the first antigen-binding domain of (1) does not substantially bind to at least one selected from human CLDN9, human CLDN4 and human CLDN3.
[6] The multispecific antigen-binding molecule of any one of [1] to [5], wherein the first antigen-binding domain of (1) does not substantially bind to a CLDN6 mutant as defined in SEQ ID NO: 53.
[7] The multispecific antigen-binding molecule of any one of [1] to [6], wherein the second antigen-binding domain in (2) binds to T-cell receptor.
[8] The multispecific antigen-binding molecule of any one of [1] to [6], wherein the second antigen-binding domain in (2) binds to CD3 epsilon chain.
[9] The multispecific antigen-binding molecule of any one of [1] to [8], wherein the first antigen-binding domain or the second antigen-binding domain is an antibody variable fragment, or both of the first and second antigen-binding domains are antibody variable fragments.
[10] The multispecific antigen-binding molecule of [9], wherein the antibody variable fragment is a Fab.
[11] The multispecific antigen-binding molecule of any one of [1] to [10], wherein the first antigen-binding domain of (1) is any one of (a1) to (a6) below:
   (a1) an antibody variable region comprising the HVR-H1 sequence of SEQ ID NO: 1, the HVR-H2 sequence of SEQ ID NO: 2, the HVR-H3 sequence of SEQ ID NO: 3, the HVR-L1 sequence of SEQ ID NO: 4, the HVR-L2 sequence of SEQ ID NO: 5, and the HVR-L3 sequence of SEQ ID NO: 6;
   (a2) an antibody variable region comprising the HVR-H1 sequence of SEQ ID NO: 9, the HVR-H2 sequence of SEQ ID NO: 10, the HVR-H3 sequence of SEQ ID NO: 11, the HVR-L1 sequence of SEQ ID NO: 12, the HVR-L2 sequence of SEQ ID NO: 13, and the HVR-L3 sequence of SEQ ID NO: 14;
   (a3) an antibody variable region comprising the HVR-H1 sequence of SEQ ID NO: 17, the HVR-H2 sequence of SEQ ID NO: 18, the HVR-H3 sequence of SEQ ID NO: 19, the HVR-L1 sequence of SEQ ID NO: 20, the HVR-L2 sequence of SEQ ID NO: 21, and the HVR-L3 sequence of SEQ ID NO: 22;
   (a4) an antibody variable region comprising the HVR-H1 sequence of SEQ ID NO: 25, the HVR-H2 sequence of SEQ ID NO: 26, the HVR-H3 sequence of SEQ ID NO: 27, the HVR-L1 sequence of SEQ ID NO: 28, the HVR-L2 sequence of SEQ ID NO: 29, and the HVR-L3 sequence of SEQ ID NO: 30;
   (a5) an antibody variable region that binds to the same epitope with any one of the antibody variable regions selected from (a1) to (a4);
   (a6) an antibody variable region that competes for the binding to human CLDN6 with any one of the antibody variable regions selected from (a1) to (a4).
[12] The multispecific antigen-binding molecule of any one of [1] to [10], wherein the first antibody variable region of (1) is any one of (b1) to (b6) below:
   (b1) an antibody variable region comprising the HVR-H1 sequence identical to the amino acid sequences of the HVR-H1 region comprised in SEQ ID NO: 7, the HVR-H2 sequence identical to the amino acid sequences of the HVR-H2 region comprised in SEQ ID NO: 7, the HVR-H3 sequence identical to the amino acid sequences of the HVR-H3 region comprised in SEQ ID NO: 7, the HVR-L1 sequence identical to the amino acid sequences of the HVR-L1 region comprised in SEQ ID NO: 8, the HVR-L2 sequence identical to the amino acid sequences of the HVR-L2 region comprised in SEQ ID NO: 8, and the HVR-L3 sequence identical to the amino acid sequences of the HVR-L3 region comprised in SEQ ID NO: 8;
   (b2) an antibody variable region comprising the HVR-H1 sequence identical to the amino acid sequences of the HVR-H1 region comprised in SEQ ID NO: 15, the HVR-H2 sequence identical to the amino acid sequences of the HVR-H2 region comprised in SEQ ID NO: 15, the HVR-H3 sequence identical to the amino acid sequences of the HVR-H3 region comprised in SEQ ID NO: 15, the HVR-L1 sequence identical to the amino acid sequences of the HVR-L1 region comprised in SEQ ID NO: 16, the HVR-L2 sequence identical to the amino acid sequences of the HVR-L2 region comprised in SEQ ID NO: 16, and the HVR-L3 sequence identical to the amino acid sequences of the HVR-L3 region comprised in SEQ ID NO: 16;
   (b3) an antibody variable region comprising the HVR-H1 sequence identical to the amino acid sequences of the HVR-H1 region comprised in SEQ ID NO: 23, the HVR-H2 sequence identical to the amino acid sequences of the HVR-H2 region comprised in SEQ ID NO: 23, the HVR-H3 sequence identical to the amino acid sequences of the HVR-H3 region comprised in SEQ ID NO: 23, the HVR-L1 sequence identical to the amino acid sequences of the HVR-L1 region comprised in SEQ ID NO: 24, the HVR-L2 sequence identical to the amino acid sequences of the HVR-L2 region comprised in SEQ ID NO: 24, and the HVR-L3 sequence identical to the amino acid sequences of the HVR-L3 region comprised in SEQ ID NO: 24;
   (b4) an antibody variable region comprising the HVR-H1 sequence identical to the amino acid sequences of the HVR-H1 region comprised in SEQ ID NO: 31, the HVR-H2 sequence identical to the amino acid sequences of the HVR-H2 region comprised in SEQ ID NO: 31, the HVR-H3 sequence identical to the amino acid sequences of the HVR-H3 region comprised in SEQ ID NO: 31, the HVR-L1 sequence identical to the amino acid sequences of the HVR-L1 region comprised in SEQ ID NO: 32, the HVR-L2 sequence identical to the amino acid sequences of the HVR-L2 region comprised in SEQ ID NO: 32, and the HVR-L3 sequence identical to the amino acid sequences of the HVR-L3 region comprised in SEQ ID NO: 32;
   (b5) an antibody variable region that binds to the same epitope with any one of the antibody variable regions selected from (b1) to (b4);
   (b6) an antibody variable region that competes for the binding to human CLDN6 with any one of the antibody variable regions selected from (b1) to (b4).
[13] The multispecific antigen-binding molecule of any one of [1] to [12], wherein the second antibody variable region of (2) is any one of (c1) to (c3) below:
   (c1) an antibody variable region comprising the HVR-H1 sequence of SEQ ID NO: 33, the HVR-H2 sequence of SEQ ID NO: 34, the HVR-H3 sequence of SEQ ID NO: 35, the HVR-L1 sequence of SEQ ID NO: 36, the HVR-L2 sequence of SEQ ID NO: 37, and the HVR-L3 sequence of SEQ ID NO: 38;
   (c2) an antibody variable region that binds to the same epitope with the antibody variable region of (c1);
   (c3) an antibody variable region that competes for the binding to CD3 epsilon chain with the antibody variable region of (c1).
[14] A multispecific antigen-binding molecule that comprises:
   (1) a first domain comprising a first antigen-binding domain binds to human CLDN6, and
   (2) a second domain comprising a second antigen-binding domain binds to T-cell receptor complex,
   wherein the first antibody variable region of (1) is any one of (a1) to (a6) below:
      (a1) an antibody variable region comprising the HVR-H1 sequence of SEQ ID NO: 1, the HVR-H2 sequence of SEQ ID NO: 2, the HVR-H3 sequence of SEQ ID NO: 3, the HVR-L1 sequence of SEQ ID NO: 4, the HVR-L2 sequence of SEQ ID NO: 5, and the HVR-L3 sequence of SEQ ID NO: 6;
      (a2) an antibody variable region comprising the HVR-H1 sequence of SEQ ID NO: 9, the HVR-H2 sequence of SEQ ID NO: 10, the HVR-H3 sequence of SEQ ID NO: 11, the HVR-L1 sequence of SEQ ID NO: 12, the HVR-L2 sequence of SEQ ID NO: 13, and the HVR-L3 sequence of SEQ ID NO: 14;
      (a3) an antibody variable region comprising the HVR-H1 sequence of SEQ ID NO: 17, the HVR-H2 sequence of SEQ ID NO: 18, the HVR-H3 sequence of SEQ ID NO: 19, the HVR-L1 sequence of SEQ ID NO: 20, the HVR-L2 sequence of SEQ ID NO: 21, and the HVR-L3 sequence of SEQ ID NO: 22;
      (a4) an antibody variable region comprising the HVR-H1 sequence of SEQ ID NO: 25, the HVR-H2 sequence of SEQ ID NO: 26, the HVR-H3 sequence of SEQ ID NO: 27, the HVR-L1 sequence of SEQ ID NO: 28, the HVR-L2 sequence of SEQ ID NO: 29, and the HVR-L3 sequence of SEQ ID NO: 30;
      (a5) an antibody variable region that binds to the same epitope with any one of the antibody variable regions selected from (a1) to (a4);
      (a6) an antibody variable region that competes for the binding to human CLDN6 with any one of the antibody variable regions selected from (a1) to (a4).
[15] A multispecific antigen-binding molecule that comprises:
   (1) a first domain comprising a first antigen-binding domain binds to human CLDN6, and
   (2) a second domain comprising a second antigen-binding domain binds to T-cell receptor complex, wherein the first antibody variable region of (1) is any one of (b 1) to (b6) below:
      (b1) an antibody variable region comprising the HVR-H1 sequence identical to the amino acid sequences of the HVR-H1 region comprised in SEQ ID NO: 7, the HVR-H2 sequence identical to the amino acid sequences of the HVR-H2 region comprised in SEQ ID NO: 7, the HVR-H3 sequence identical to the amino acid sequences of the HVR-H3 region comprised in SEQ ID NO: 7, the HVR-L1 sequence identical to the amino acid sequences of the HVR-L1 region comprised in SEQ ID NO: 8, the HVR-L2 sequence identical to the amino acid sequences of the HVR-L2 region comprised in SEQ ID NO: 8, and the HVR-L3 sequence identical to the amino acid sequences of the HVR-L3 region comprised in SEQ ID NO: 8;
      (b2) an antibody variable region comprising the HVR-H1 sequence identical to the amino acid sequences of the HVR-H1 region comprised in SEQ ID NO: 15, the HVR-H2 sequence identical to the amino acid sequences of the HVR-H2 region comprised in SEQ ID NO: 15, the HVR-H3 sequence identical to the amino acid sequences of the HVR-H3 region comprised in SEQ ID NO: 15, the HVR-L1 sequence identical to the amino acid sequences of the HVR-L1 region comprised in SEQ ID NO: 16, the HVR-L2 sequence identical to the amino acid sequences of the HVR-L2 region comprised in SEQ ID NO: 16, and the HVR-L3 sequence identical to the amino acid sequences of the HVR-L3 region comprised in SEQ ID NO: 16;
      (b3) an antibody variable region comprising the HVR-H1 sequence identical to the amino acid sequences of the HVR-H1 region comprised in SEQ ID NO: 23, the HVR-H2 sequence identical to the amino acid sequences of the HVR-H2 region comprised in SEQ ID NO: 23, the HVR-H3 sequence identical to the amino acid sequences of the HVR-H3 region comprised in SEQ ID NO: 23, the HVR-L1 sequence identical to the amino acid sequences of the HVR-L1 region comprised in SEQ ID NO: 24, the HVR-L2 sequence identical to the amino acid sequences of the HVR-L2 region comprised in SEQ ID NO: 24, and the HVR-L3 sequence identical to the amino acid sequences of the HVR-L3 region comprised in SEQ ID NO: 24;
      (b4) an antibody variable region comprising the HVR-H1 sequence identical to the amino acid sequences of the HVR-H1 region comprised in SEQ ID NO: 31, the HVR-H2 sequence identical to the amino acid sequences of the HVR-H2 region comprised in SEQ ID NO: 31, the HVR-H3 sequence identical to the amino acid sequences of the HVR-H3 region comprised in SEQ ID NO: 31, the HVR-L1 sequence identical to the amino acid sequences of the HVR-L1 region comprised in SEQ ID NO: 32, the HVR-L2 sequence identical to the amino acid sequences of the HVR-L2 region comprised in SEQ ID NO: 32, and the HVR-L3 sequence identical to the amino acid sequences of the HVR-L3 region comprised in SEQ ID NO: 32;
      (b5) an antibody variable region that binds to the same epitope with any one of the antibody variable regions selected from (b1) to (b4);
      (b6) an antibody variable region that competes for the binding to human CLDN6 with any one of the antibody variable regions selected from (b1) to (b4).
[16] The multispecific antigen-binding molecule of [14] or [15], wherein the multispecific antigen-binding molecule has cytotoxic activity.
[17] The multispecific antigen-binding molecule of [16], wherein the cytotoxic activity is T-cell-dependent cytotoxic activity.
[18] The multispecific antigen-binding molecule of any one of [14] to [17], wherein the first antigen-binding domain of (1) specifically binds to human CLDN6.
[19] The multispecific antigen-binding molecule of any one of [14] to [18], wherein the first antigen-binding domain (1) does not substantially bind to at least one selected from human CLDN9, human CLDN4 and human CLDN3.
[20] The multispecific antigen-binding molecule of any one of [14] to [19], wherein the first antigen-binding domain of (1) does not substantially bind to a CLDN6 mutant as defined in SEQ ID NO: 53.
[21] The multispecific antigen-binding molecule of any one of [14] to [20], wherein the second antigen-binding domain of (2) binds to T-cell receptor.
[22] The multispecific antigen-binding molecule of any one [14] to [20], wherein the second antigen-binding domain of (2) binds to CD3 epsilon chain.
[23] The multispecific antigen-binding molecule of any one of [14] to [22], wherein the first antigen-binding domain or the second antigen-binding domain comprises an antibody variable fragment.
[24] The multispecific antigen-binding molecule of [23], wherein the antibody variable fragment is a Fab.
[25] The multispecific antigen-binding molecule of any one of [14] to [24], wherein the second antigen-binding domain of (2) is any one of (c1) to (c3) below:
   (c1) an antibody variable region comprising the HVR-H1 sequence of SEQ ID NO: 33, the HVR-H2 sequence of SEQ ID NO: 34, the HVR-H3 sequence of SEQ ID NO: 35, the HVR-L1 sequence of SEQ ID NO: 36, the HVR-L2 sequence of SEQ ID NO: 37, and the HVR-L3 sequence of SEQ ID NO: 38;
   (c2) an antibody variable region that binds to the same epitope with the antibody variable region of (c1);
   (c3) an antibody variable region that competes for the binding to CD3 epsilon chain with the antibody variable region of (c1).
[26] The multispecific antigen-binding molecule of any one of [1] to [13], wherein the Fc region of (3) is an Fc region with reduced binding activity towards an Fc gamma receptor.
[27] The multispecific antigen-binding molecule of [26], wherein the Fc region of is an Fc region with at least one amino acid mutation at any of the Fc region-constituting amino acids of SEQ ID NOs: 62 to 65 (IgG1 to IgG4).
[28] The multispecific antigen-binding molecule of [26] or [27], wherein the Fc region is an Fc region with mutation of at least one amino acid selected from the following amino acid positions specified by EU numbering:
   position 220, position 226, position 229, position 231, position 232, position 233, position 234, position 235, position 236, position 237, position 238, position 239, position 240, position 264, position 265, position 266, position 267, position 269, position 270, position 295, position 296, position 297, position 298, position 299, position 300, position 325, position 327, position 328, position 329, position 330, position 331, and position 332.
[29] The multispecific antigen-binding molecule of any one of [1] to [28], wherein the multispecific antigen-binding molecule is a bispecific antibody.
[30] Isolated nucleic acid encoding the multispecific antigen-binding molecule of any one of [1] to [28] or the bispecific antibody of [29].
[31] A host cell comprising the nucleic acid of [30].
[32] A method of producing a multispecific antigen-binding molecule or bispecific antibody comprising culturing the host cell of [31] so that the multispecific antigen-binding molecule or bispecific antibody is produced.
[33] The method of [32], further comprising recovering the multispecific antigen-binding molecule or bispecific antibody from the host cell.
[34] A pharmaceutical composition comprising the multispecific antigen-binding molecule of any one of [1] to [28], or the bispecific antibody of [29], and a pharmaceutically acceptable carrier.
[35] The pharmaceutical composition of [34], which induces T-cell-dependent cytotoxicity.
[36] The multispecific antigen-binding molecule of any one of [1] to [28] or the bispecific antibody of [29] for use as a medicament.
[37] The multispecific antigen-binding molecule of any one of [1] to [28] or the bispecific antibody of [29] for use in treating cancer.
[38] The multispecific antigen-binding molecule of any one of [1] to [28] or the bispecific antibody of [29] for use in treating ovarian cancer, non-small cell lung cancer, gastric cancer, or liver cancer.
[39] Use of the multispecific antigen-binding molecule of any one of [1] to [28] or the bispecific antibody of [29] in the manufacture of a medicament.
[40] Use of the multispecific antigen-binding molecule of any one of [1] to [28] or the bispecific antibody of [29] in the manufacture of a medicament for treatment of cancer.
[41] Use of the multispecific antigen-binding molecule of any one of [1] to [28] or the bispecific antibody of [29] in the manufacture of a medicament for treatment of ovarian cancer, non-small cell lung cancer, gastric cancer, or liver cancer.
[42] A method of treating an individual having cancer comprising administering to the individual an effective amount of multispecific antigen-binding molecule of any one of [1] to [28] or the bispecific antibody of [29].
[43] A method of treating an individual having ovarian cancer, non-small cell lung cancer, gastric cancer, or liver cancer comprising administering to the individual an effective amount of multispecific antigen-binding molecule of any one of [1] to [28] or the bispecific antibody of [29].
[44] A kit comprising the multispecific antigen-binding molecule of any one of [1] to [28] or the bispecific antibody of [29], and instructions for use.

### Advantageous Effects of the Invention

The present invention provides multispecific antigen-binding molecules that enable cancer treatment by having T-cells close to CLDN6-expressing cells and using the cytotoxicity of T-cells against the CLDN6-expressing cancer cells, methods for producing the multispecific antigen-binding molecules, and therapeutic agents containing such a multispecific antigen-binding molecule as an active ingredient for inducing cellular cytotoxicity, as a new approach of cancer treatment. Multispecific antigen-binding molecules of the present invention have strong anti-tumor activity, inducing cellular cytotoxicity, and can target and damage CLDN6-expressing cells, thus enable treatment and prevention of various cancers. Furthermore, the multispecific antigen-binding molecules of the present invention have a long half-life in blood, as well as excellent safety properties that result in no induction of cancer antigen-independent cytokine storm or such. This allows desirable treatments that are highly safe and convenient, and reduces the physical burden for patients.

### BRIEF DESCRIPTION OF DRAWINGS

FIG. 1, Binding activities of the CLDN6-TRABs to various Ba/F3 tranfectants. The vertical axis indicates the Geo-mean value of an antibody against a Claudin expressing Ba/F3 cell, in a logarithmic manner, taken as binding activity.

FIG. 2, Amino acid sequence alignment of human CLDN9 and human CLDN6. Human CLDN9 and human CLDN6 comprises almost the same sequence in extracellular domain 1, except the N-terminus residue (Met/Leu at position 29). Two amino acids in extracellular domain 2 are different in human CLDN9 and human CLDN6 (Arg/Leu at position 145 and Gln/Leu at position 156).

FIG. 3, Cytotoxic activity of the CLDN6-TRABs evaluated by lactate dehydrogenase (LDH) release assay. The horizontal axis indicates the concentration of CLDN6-TRABs.

FIG. 4-1, T cell activation mediated by the CLDN6-TRABs. The horizontal axis indicates the concentration of CLDN6-TRABs, and the vertical axis indicates the luminescence fold of antibody containing well compared with well without antibody. Cancer cell lines ABC-1 (FIG. 4A), PA-1 (FIG. 4B), OV-90 (FIG. 4C), HuH-7 (FIG. 4D), SCH (FIG. 4E) and Ba/F3 transfectants BaF3_hCLDN6 (FIG. 4F), BaF3_hCLDN9 (FIG. 4G), BaF3_hCLDN6(Q156L) (FIG. 4H) were used as target cells.

FIG. 4-2, Continuation of FIG. 4-1.

FIG. 4-3, Continuation of FIG. 4-2.

FIG. 4-4, Continuation of FIG. 4-3.

FIG. 5, T cell dependent cell growth inhibition mediated by the CLDN6-TRABs. The horizontal axis indicates the concentration of CLDN6-TRABs, and the vertical axis indicates the rate of cell growth inhibition. Cancer cell lines HuH-7 (FIG. 5A) and OV-90 (FIG. 5B) were used as target cells.

FIG. 6, In vivo anti-tumor efficacy of the CLDN6-TRABs. FIG. 6A shows the tumor volume change in HuH-7 T cell injection model. FIG. 6B shows the tumor volume change in OV-90 T cell injection model.

FIG. 7, The temporal change of the concentration of CLDND6-TRABs in HuH-7 transplant mice (FIG. 7A) and OV-90 transplant mice (FIG. 7B). The vertical axis indicates the antibody concentration (micro g/mL) in the mouse plasma.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present invention illustrated herein.

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Antigen-Binding Molecule

The term “antigen-binding molecules”, as used herein, refers to any molecule that comprises an antigen-binding domain or any molecule that has binding activity to an antigen, and may further refers to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such.

A favorable example of an antigen-binding molecule of the present invention is an antigen-binding molecule that comprises a plurality of antigen-binding domains. In certain embodiments, the antigen-binding molecule of the present invention is an antigen-binding molecule that comprises two antigen-binding domains with different antigen-binding specificities. In certain embodiments, the antigen-binding molecule of the present invention is an antigen-binding molecule that comprises two antigen-binding molecules comprising two antigen-binding domains with different antigen-binding specificities, and an FcRn-binding domain contained in an antibody Fc region. As a method for extending the blood half-life of a protein administered to a living body, the method of adding an FcRn-binding domain of an antibody to the protein of interest and utilizing the function of FcRn-mediated recycling is well known.

### Antigen-Binding Domain

The term “antigen-binding domain”, as used herein, refers to an antibody portion which comprises a region that specifically binds and is complementary to the whole or a portion of an antigen. When the molecular weight of an antigen is large, an antibody can only bind to a particular portion of the antigen. The particular portion is called “epitope”. An antigen-binding domain can be provided from one or more antibody variable domains. Preferably, the antigen-binding domains contain antibody variable region that comprising both the antibody light chain variable region (VL) and antibody heavy chain variable region (VH). Such preferable antigen-binding domains include, for example, “single-chain Fv (scFv)”, “single-chain antibody”, “Fv”, “single-chain Fv₂ (scFv₂)”, “Fab”, and “F (ab′)₂”.

The antigen-binding domains of antigen-binding molecules of the present invention may bind to the same epitope. The epitope can be present in a protein comprising the amino acid sequence of SEQ ID NO: 44, 45 or 61. Alternatively, the antigen-binding domains of polypeptide complexes of the present invention may individually bind to different epitopes. The epitope can be present in a protein comprising the amino acid sequence of SEQ ID NO: 44, 45 or 61.

The antigen-binding domain of an antigen-binding molecule of the present invention “binds to CLDN6 or T cell receptor complex”. That is, CLDN6 and a T cell receptor complex are preferable antigens of interest, and the antigen-binding domain has binding activity to its antigen of interest. As used herein, the phrase “has binding activity” refers to the activity of an antigen-binding domain, antibody, antigen-binding molecule, antibody variable fragment, or such (hereinafter, “antigen-binding domain or such”) to bind to an antigen of interest at a level of specific binding higher than the level of non-specific or background binding. In other words, such an antigen-binding domain or such “has a specific/significant binding activity” towards the antigen of interest. The specificity can be measured by any methods for detecting affinity or binding activity as mentioned herein or known in the art. The above-mentioned level of specific binding may be high enough to be recognized by a skilled person as being significant. For example, when a skilled person can detect or observe any significant or relatively strong signals or values of binding between the antigen-binding domain or such and the antigen of interest in a suitable binding assay, it can be said that the antigen-binding domain or such has a “specific/significant binding activity” towards the antigen of interest. Alternatively, “have a specific/significant binding activity” can be rephrased as “specifically/significantly bind” (to the antigen of interest). Sometimes, the phrase “having binding activity” has substantially the same meaning as the phrase “having a specific/significant binding activity” in the art.

### Claudin-6 (CLDN6)

The term “CLDN6”, as used herein, refers to any native Claudin-6 from any vertebrate source, including mammals such as primates (e.g. humans) and rodents (e.g., mice and rats), unless otherwise indicated.

### Affinity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

### Methods to Determine Affinity

In certain embodiments, the antigen-binding domain of an antigen-binding molecule or antibody provided herein has a dissociation constant (Kd) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸M or less, 10⁻⁸M to 10⁻¹³M, 10⁻⁹M to 10⁻¹³M) for its antigen. In certain embodiments, the Kd value of the first antigen-binding domain of the antibody/antigen-binding molecule for CLDN6 falls within the range of 1-40, 1-50, 1-70, 1-80, 30-50, 30-70, 30-80, 40-70, 40-80, or 60-80 nM.

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro l/well of scintillant (MICROSCINT-20™; Packard) is added, and the plates are counted on a TOPCOUNT′ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, N.J.) is performed at 25 degrees C. with immobilized antigen CMS chips at ˜10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 micro g/ml (˜0.2 micro M) before injection at a flow rate of 5 micro l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro l/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

Methods for measuring the affinity of the antigen-binding domain of an antibody are described above, and one skilled in art can carry out affinity measurement for other antigen-binding domains.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

### HVR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).
   Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 is also mentioned as “HCDR1”, “HCDR2”, “HCDR3”, LCDR1”, “LCDR2”, and “LCDR3”, respectively.

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### Identity (Sequence Identity)

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

Methods for Producing an Antibody with Desired Binding Activity

Methods for producing an antibody with desired binding activity are known to those skilled in the art. Below is an example that describes a method for producing an antibody (anti-CLDN6 antibody) that binds to Claudin 6 (hereinafter, also referred to as CLDN6). Antibodies that bind to a T-cell receptor complex and so on can also be produced according to the example described below.

Anti-CLDN6 antibodies can be obtained as polyclonal or monoclonal antibodies using known methods. The anti-CLDN6 antibodies preferably produced are monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a CLDN6 protein as a sensitizing antigen. Resulting immune cells are fused with known parental cells by conventional cell fusion methods. Then, hybridomas producing an anti-CLDN6 antibody can be selected by screening for monoclonal antibody-producing cells using conventional screening methods.

Specifically, monoclonal antibodies are prepared as mentioned below. First, the CLDN6 gene whose nucleotide sequence is disclosed in RefSeq Accession No. NM 021195.4 or BC008934.2 can be expressed to produce the CLDN6 protein shown in Genbank Accession No. NP 067018.2 (SEQ ID NO: 45) or AAH08934.1 (SEQ ID NO: 44), which will be used as a sensitizing antigen for antibody preparation. Alternatively, a nucleotide encoding the first or second extracellular domain (ECD) of CLDN6 can be expressed to produce a CLDN6 ECD-containing protein. That is, a gene sequence encoding full-length CLDN6 or CLDN6 ECD is inserted into a known expression vector, and appropriate host cells are transformed with this vector. Either the first or the second extracellular domain of CLDN6 could be used. The desired human full-length CLDN6 or CLDN6 ECD protein is purified from the host cells or their culture supernatants by known methods. Alternatively, it is possible to use a purified natural CLDN6 protein as a sensitizing antigen.

The purified full-length CLDN6 or CLDN6 ECD protein can be used as a sensitizing antigen for use in immunization of mammals. Partial peptides of full-length CLDN6 or CLDN6 ECD can also be used as sensitizing antigens. In this case, the partial peptides may also be obtained by chemical synthesis from the human CLDN6 amino acid sequence. Furthermore, they may also be obtained by incorporating a portion of the CLDN6 gene into an expression vector and expressing it. Moreover, they may also be obtained by degrading the CLDN6 protein using proteases, but the region and size of the CLDN6 peptide used as the partial peptide are not particularly limited to a special embodiment. As the preferred region, any sequence from the amino acid sequence corresponding to the amino acids at positions 29 to 81 or the amino acids at positions 138 to 159 in the amino acid sequence of SEQ ID NO: 44 or 45 may be selected. The number of amino acids constituting a peptide to be used as the sensitizing antigen is at least five or more, or preferably for example, six or more, or seven or more. More specifically, peptides consisting of 8 to 50 residues or preferably 10 to 30 residues may be used as the sensitizing antigen.

For sensitizing antigen, alternatively it is possible to use a fusion protein prepared by fusing a desired partial polypeptide or peptide of the full-length CLDN6 or CLDN6 ECD protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing CLDN6 to be used as a sensitizing antigen, and immunization methods using CLDN6 are also described in the Examples of this specification later.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as CLDN6; and
there is no need to purify the antigen for immunization.

In order to prepare a monoclonal antibody of the present invention using DNA immunization, first, a DNA expressing a CLDN6 protein is administered to an animal to be immunized. The CLDN6-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized. Antibodies that recognized CLDN6 can also be produced by the methods described in WO 2003/104453.

After immunizing a mammal as described above, an increase in the titer of a CLDN6-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:

P3(P3x63Ag8.653) (J. Immunol. (1979) 123 (4), 1548-1550);

P3x63Ag8U.1 (Current Topics in Microbiology and Immunology (1978)81, 1-7);

NS-1 (C. Eur. J. Immunol. (1976)6 (7), 511-519);

MPC-11 (Cell (1976) 8 (3), 405-415);

SP2/0 (Nature (1978) 276 (5685), 269-270);

FO (J. Immunol. Methods (1980) 35 (1-2), 1-21);

S194/5.XX0.BU.1 (J. Exp. Med. (1978) 148 (1), 313-323);

R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46). More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37 degrees C. is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, a CLDN6-binding monoclonal antibody can bind to CLDN6 expressed on the cell surface. Such a monoclonal antibody can be screened by fluorescence activated cell sorting (FACS). FACS is a system that assesses the binding of an antibody to cell surface by analyzing cells contacted with a fluorescent antibody using laser beam, and measuring the fluorescence emitted from individual cells.

To screen for hybridomas that produce a monoclonal antibody of the present invention by FACS, CLDN6-expressing cells are first prepared. Cells preferably used for screening are mammalian cells in which CLDN6 is forcedly expressed. As control, the activity of an antibody to bind to cell-surface CLDN6 can be selectively detected using non-transformed mammalian cells as host cells. Specifically, hybridomas producing an anti-CLDN6 monoclonal antibody can be isolated by selecting hybridomas that produce an antibody which binds to cells forced to express CLDN6, but not to host cells.

Alternatively, the activity of an antibody to bind to immobilized CLDN6-expressing cells can be assessed based on the principle of ELISA. For example, CLDN6-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Preferably, the present invention provides nucleic acids that encode a multispecific antigen-binding molecule of the present invention. The present invention also provides a vector into which the nucleic acid encoding the multispecific antigen-binding molecule is introduced, i.e., a vector comprising the nucleic acid. Furthermore, the present invention provides a cell comprising the nucleic acid or the vector. The present invention also provides a method for producing the multispecific antigen-binding molecule by culturing the cell. The present invention further provides multispecific antigen-binding molecules produced by the method.

For example, a cDNA encoding the variable region (V region) of an anti-CLDN6 antibody is prepared from hybridoma cells expressing the anti-CLDN6 antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gamma1, gamma2a, gamma2b, and gamma3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the CLDN6-binding activity of a reshaped immunoglobulin as an indicator. For example, when the objective is to isolate an antibody against CLDN6, it is more preferred that the binding of the antibody to CLDN6 is specific. A CLDN6-binding antibody can be screened, for example, by the following steps:
(1) contacting a CLDN6-expressing cell with an antibody comprising the V region encoded by a cDNA isolated from a hybridoma;
(2) detecting the binding of the antibody to the CLDN6-expressing cell; and
(3) selecting an antibody that binds to the CLDN6-expressing cell.

Methods for detecting the binding of an antibody to CLDN6-expressing cells are known. Specifically, the binding of an antibody to CLDN6-expressing cells can be detected by the above-described techniques such as FACS. Immobilized samples of CLDN6-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having a desired binding activity.

After isolation of the cDNA encoding the V region of the anti-CLDN6 antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-CLDN6 antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in frame the two genes digested with the same combination of restriction enzymes.

To produce an anti-CLDN6 monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. In the Examples described below, a peptide having the amino acid sequence MGWSCIILFLVATATGVHS (SEQ ID NO: 79) is used as a signal sequence. Meanwhile, other appropriate signal sequences may be attached. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-CLDN6 antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. All of these expression systems are applicable to isolation of domains including antibody variable regions of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells: yeasts: the Saccharomyces genus such as Saccharomyces cerevisiae, and the Pichia genus such as Pichia pastoris; and filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat beta-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

### Methods for Producing a Humanized Antibody

When an antigen-binding molecule described herein is administered to human, a domain derived from a genetically recombinant antibody that has been artificially modified to reduce the heterologous antigenicity against human and such, can be appropriately used as the domain of the antigen-binding molecule including an antibody variable region. Such genetically recombinant antibodies include, for example, humanized antibodies. These modified antibodies are appropriately produced by known methods. Furthermore, generally, the binding specificity of a certain antibody can be introduced into another antibody by CDR grafting.

Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length V region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856)

### Methods for Producing a Human Antibody

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Epitope

“Epitope” means an antigenic determinant in an antigen, and refers to an antigen site to which the antigen-binding domain of an antigen-binding molecule or antibody disclosed herein binds. Thus, for example, the epitope can be defined according to its structure. Alternatively, the epitope may be defined according to the antigen-binding activity of an antigen-binding molecule or antibody that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antigen-binding domain recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antigen-binding domain. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test antigen-binding molecule or antibody containing an anti-CLDN6 antigen-binding domain are described below. According to the examples below, methods for assessing the epitope binding by a test antigen-binding molecule or antibody containing an antigen-binding domain for an antigen other than CLDN6, can also be appropriately conducted.

For example, whether a test antigen-binding molecule or antibody containing an anti-CLDN6 antigen-binding domain recognizes a linear epitope in the CLDN6 molecule can be confirmed for example as mentioned below. A linear peptide comprising an amino acid sequence forming the extracellular domain of CLDN6 is synthesized for the above purpose. The peptide can be synthesized chemically, or obtained by genetic engineering techniques using a region encoding the amino acid sequence corresponding to the extracellular domain in a CLDN6 cDNA. Then, a test antigen-binding molecule or antibody containing an anti-CLDN6 antigen-binding domain is assessed for its binding activity towards a linear peptide comprising the amino acid sequence forming the extracellular domain. For example, an immobilized linear peptide can be used as an antigen by ELISA to evaluate the binding activity of the polypeptide complex towards the peptide. Alternatively, the binding activity towards a linear peptide can be assessed based on the level that the linear peptide inhibits the binding of the antigen-binding molecule or antibody to CLDN6-expressing cells. These tests can demonstrate the binding activity of the antigen-binding molecule or antibody towards the linear peptide.

Whether a test antigen-binding molecule or antibody containing an anti-CLDN6 antigen-binding domain recognizes a conformational epitope can be assessed as follows. CLDN6-expressing cells are prepared for the above purpose. A test antigen-binding molecule or antibody containing an anti-CLDN6 antigen-binding domain can be determined to recognize a conformational epitope when it strongly binds to CLDN6-expressing cells upon contact, but does not substantially bind to an immobilized linear peptide comprising an amino acid sequence forming the extracellular domain of CLDN6. Herein, “not substantially bind” means that the binding activity is 80% or less, generally 50% or less, preferably 30% or less, and particularly preferably 15% or less compared to the binding activity towards cells expressing CLDN6.

Methods for assaying the binding activity of a test antigen-binding molecule or antibody containing an anti-CLDN6 antigen-binding domain towards CLDN6-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using CLDN6-expressing cells as antigen.

In the ELISA format, the binding activity of a test antigen-binding molecule or antibody containing an anti-CLDN6 antigen-binding domain towards CLDN6-expressing cells can be assessed quantitatively by comparing the levels of signal generated by enzymatic reaction. Specifically, a test polypeptide complex is added to an ELISA plate onto which CLDN6-expressing cells are immobilized. Then, the test antigen-binding molecule or antibody bound to the cells is detected using an enzyme-labeled antibody that recognizes the test antigen-binding molecule or antibody. Alternatively, when FACS is used, a dilution series of a test antigen-binding molecule or antibody is prepared, and the antibody binding titer for CLDN6-expressing cells can be determined to compare the binding activity of the test antigen-binding molecule or antibody towards CLDN6-expressing cells.

The binding of a test antigen-binding molecule or antibody towards an antigen expressed on the surface of cells suspended in buffer or the like can be detected using a flow cytometer. Known flow cytometers include, for example, the following devices:

FACSCanto™ II

FACSAria™

FACSArray™

FACSVantage™ SE

FACSCalibur™ (all are trade names of BD Biosciences)

EPICS ALTRA HyPerSort

Cytomics FC 500

EPICS XL-MCL ADC EPICS XL ADC

Cell Lab Quanta/Cell Lab Quanta SC (all are trade names of Beckman Coulter)

Preferable methods for assaying the binding activity of a test antigen-binding molecule or antibody containing an anti-CLDN6 antigen-binding domain towards an antigen include, for example, the following method. First, CLDN6-expressing cells are reacted with a test antigen-binding molecule or antibody, and then this is stained with an FITC-labeled secondary antibody that recognizes the antigen-binding molecule or antibody. The test antigen-binding molecule or antibody is appropriately diluted with a suitable buffer to prepare the antigen-binding molecule or antibody at a desired concentration. For example, the antigen-binding molecule or antibody can be used at a concentration within the range of 10 micro g/ml to 10 ng/ml. Then, the fluorescence intensity and cell count are determined using FACSCalibur (BD). The fluorescence intensity obtained by analysis using the CELL QUEST Software (BD), i.e., the Geometric Mean value, reflects the quantity of antibody bound to cells. That is, the binding activity of a test antigen-binding molecule or antibody, which is represented by the quantity of the test antigen-binding molecule or antibody bound, can be determined by measuring the Geometric Mean (Geo-mean) value.

Whether a test antigen-binding molecule or antibody containing an anti-CLDN6 antigen-binding domain shares a common epitope with another antigen-binding molecule or antibody can be assessed based on the competition between the two antigen-binding molecules or antibodies for the same epitope. The competition between the antigen-binding molecules or antibodies can be detected by cross-blocking assay or the like. For example, the competitive ELISA assay is a preferred cross-blocking assay.

Specifically, in cross-blocking assay, the CLDN6 protein immobilized to the wells of a microtiter plate is pre-incubated in the presence or absence of a candidate competitor antigen-binding molecule or antibody, and then a test antigen-binding molecule or antibody is added thereto. The quantity of test antigen-binding molecule or antibody bound to the CLDN6 protein in the wells is indirectly correlated with the binding ability of a candidate competitor antigen-binding molecule or antibody that competes for the binding to the same epitope. That is, the greater the affinity of the competitor antigen-binding molecule or antibody for the same epitope, the lower the binding activity of the test antigen-binding molecule or antibody towards the CLDN6 protein-coated wells.

The quantity of the test antigen-binding molecule or antibody bound to the wells via the CLDN6 protein can be readily determined by labeling the antigen-binding molecule or antibody in advance. For example, a biotin-labeled antigen-binding molecule or antibody is measured using an avidin/peroxidase conjugate and appropriate substrate. In particular, cross-blocking assay that uses enzyme labels such as peroxidase is called “competitive ELISA assay”. The antigen-binding molecule or antibody can also be labeled with other labeling substances that enable detection or measurement. Specifically, radiolabels, fluorescent labels, and such are known.

When the candidate competitor antigen-binding molecule or antibody can block the binding by a test antigen-binding molecule or antibody containing an anti-CLDN6 antigen-binding domain by at least 20%, preferably at least 20 to 50%, and more preferably at least 50% compared to the binding activity in a control experiment conducted in the absence of the competitor antigen-binding molecule or antibody, the test antigen-binding molecule or antibody is determined to substantially bind to the same epitope bound by the competitor antigen-binding molecule or antibody, or compete for the binding to the same epitope.

When the structure of an epitope bound by a test antigen-binding molecule or antibody containing an anti-CLDN6 antigen-binding domain has already been identified, whether the test and control antigen-binding molecules or antibodies share a common epitope can be assessed by comparing the binding activities of the two antigen-binding molecules or antibodies towards a peptide prepared by introducing amino acid mutations into the peptide forming the epitope.

To measure the above binding activities, for example, the binding activities of test and control antigen-binding molecules or antibodies towards a linear peptide into which a mutation is introduced are compared in the above ELISA format. Besides the ELISA methods, the binding activity towards the mutant peptide bound to a column can be determined by flowing test and control antigen-binding molecules or antibodies in the column, and then quantifying the antigen-binding molecule or antibody eluted in the elution solution. Methods for adsorbing a mutant peptide to a column, for example, in the form of a GST fusion peptide, are known.

Alternatively, when the identified epitope is a conformational epitope, whether test and control antigen-binding molecules or antibodies share a common epitope can be assessed by the following method. First, CLDN6-expressing cells and cells expressing CLDN6 with a mutation introduced into the epitope are prepared. The test and control antigen-binding molecules or antibodies are added to a cell suspension prepared by suspending these cells in an appropriate buffer such as PBS. Then, the cell suspensions are appropriately washed with a buffer, and an FITC-labeled antibody that recognizes the test and control antigen-binding molecules or antibodies is added thereto. The fluorescence intensity and number of cells stained with the labeled antibody are determined using FACSCalibur (BD). The test and control antigen-binding molecules or antibodies are appropriately diluted using a suitable buffer, and used at desired concentrations. For example, they may be used at a concentration within the range of 10 micro g/ml to 10 ng/ml. The fluorescence intensity determined by analysis using the CELL QUEST Software (BD), i.e., the Geometric Mean value, reflects the quantity of labeled antibody bound to cells. That is, the binding activities of the test and control antigen-binding molecules or antibodies, which are represented by the quantity of labeled antibody bound, can be determined by measuring the Geometric Mean value.

In the above method, whether an antigen-binding molecule or antibody does “not substantially bind to cells expressing mutant CLDN6” can be assessed, for example, by the following method. First, the test and control antigen-binding molecules or antibodies bound to cells expressing mutant CLDN6 are stained with a labeled antibody. Then, the fluorescence intensity of the cells is determined. When FACSCalibur is used for fluorescence detection by flow cytometry, the determined fluorescence intensity can be analyzed using the CELL QUEST Software. From the Geometric Mean values in the presence and absence of the antigen-binding molecule or antibody, the comparison value (delta Geo-Mean) can be calculated according to the following formula to determine the ratio of increase in fluorescence intensity as a result of the binding by the antigen-binding molecule or antibody.

delta Geo-Mean=Geo-Mean (in the presence of the antigen-binding molecule or antibody)/Geo-Mean (in the absence of the antigen-binding molecule or antibody)

The Geometric Mean comparison value (delta Geo-Mean value for the mutant CLDN6 molecule) determined by the above analysis, which reflects the quantity of a test antigen-binding molecule or antibody bound to cells expressing mutant CLDN6, is compared to the delta Geo-Mean comparison value that reflects the quantity of the test antigen-binding molecule or antibody bound to CLDN6-expressing cells. In this case, the concentrations of the test antigen-binding molecule or antibody used to determine the delta Geo-Mean comparison values for CLDN6-expressing cells and cells expressing mutant CLDN6 are particularly preferably adjusted to be equal or substantially equal. An antigen-binding molecule or antibody that has been confirmed to recognize an epitope in CLDN6 is used as a control antigen-binding molecule or antibody.

If the delta Geo-Mean comparison value of a test antigen-binding molecule or antibody for cells expressing mutant CLDN6 is smaller than the delta Geo-Mean comparison value of the test antigen-binding molecule or antibody for CLDN6-expressing cells by at least 80%, preferably 50%, more preferably 30%, and particularly preferably 15%, then the test antigen-binding molecule or antibody “does not substantially bind to cells expressing mutant CLDN6”. The formula for determining the Geo-Mean (Geometric Mean) value is described in the CELL QUEST Software User's Guide (BD biosciences). When the comparison shows that the comparison values are substantially equivalent, the epitope for the test and control antigen-binding molecules or antibodies can be determined to be the same.

### Specificity

“Specific” means that a molecule that binds specifically to one or more binding partners does not show any significant binding to molecules other than the partners. Furthermore, “specific” is also used when an antigen-binding domain is specific to a particular epitope of multiple epitopes contained in an antigen. When an epitope bound by an antigen-binding domain is contained in multiple different antigens, an antigen-binding molecule containing the antigen-binding domain can bind to various antigens that have the epitope.

### Monospecific Antigen-Binding Molecules

The term “monospecific antigen-binding molecule” is used to refer to antigen-binding molecules that specifically bind to only one type of antigen. A favorable example of a monospecific antigen-binding molecule is an antigen-binding molecule that comprises a single type of antigen-binding domain. Monospecific antigen-binding molecules can comprise a single antigen-binding domain or a plurality of antigen-binding domains of the same type. A favorable example of monospecific antigen-binding molecules is a monospecific antibody. When the monospecific antigen-binding molecule is a monospecific antibody of the IgG form, the monospecific antibody comprises two antibody variable fragments that have the same antigen-binding specificity.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

### Variable Fragment (Fv)

Herein, the term “variable fragment (Fv)” refers to the minimum unit of an antibody-derived antigen-binding domain that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)₂

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an antigen-binding domain in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)2 is a single-chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)2 preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, the form of an antigen-binding domain forming an sc(Fv)₂ include an antibody in which the two VH units and two VL units are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.

[VL]-linker-[VH]-linker-[VH]-linker-[VL]

[VH]-linker-[VL]-linker-[VL]-linker-[VH]

[VH]-linker-[VH]-linker-[VL]-linker-[VL]

[VL]-linker-[VL]-linker-[VH]-linker-[VH]

[VL]-linker-[VH]-linker-[VL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present invention may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present invention. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser
Gly Ser
Gly Gly Ser
Ser Gly Gly
Gly Gly Gly Ser (SEQ ID NO: 66)
Ser Gly Gly Gly (SEQ ID NO: 67)
Gly Gly Gly Gly Ser (SEQ ID NO: 68)
Ser Gly Gly Gly Gly (SEQ ID NO: 69)
Gly Gly Gly Gly Gly Ser (SEQ ID NO: 70)
Ser Gly Gly Gly Gly Gly (SEQ ID NO: 71)
Gly Gly Gly Gly Gly Gly Ser (SEQ ID NO: 72)
Ser Gly Gly Gly Gly Gly Gly (SEQ ID NO: 73)
(Gly Gly Gly Gly Ser (SEQ ID NO: 68))n
(Ser Gly Gly Gly Gly (SEQ ID NO: 69))n

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:

N-hydroxy succinimide (NETS),

disuccinimidyl suberate (DSS),

bis(sulfosuccinimidyl) suberate (BS3),

dithiobis(succinimidyl propionate) (DSP),

dithiobis(sulfosuccinimidyl propionate) (DTSSP),

ethylene glycol bis(succinimidyl succinate) (EGS),

ethylene glycol bis(sulfosuccinimidyl succinate) (sulfo-EGS),

disuccinimidyl tartrate (DST), di sulfosuccinimidyl tartrate (sulfo-DST),

bis[2-(succinimidoxycarbonyloxy)ethyl] sulfone (BSOCOES), and

bis[2-(sulfosuccinimidoxycarbonyloxy)ethyl] sulfone (sulfo-BSOCOES). These

crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)2” consists of two light chains and two heavy chains comprising the constant region of a CH1 domain and a portion of CH2 domains so that disulfide bonds are formed between the two heavy chains. The F(ab′)2 disclosed herein can be preferably produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Fc Region

The term “Fc region” or “Fc domain” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RITA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma Rib and Fc gamma RIc; Fc gamma RH (CD32) including isoforms Fc gamma RIIA (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIM (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RH (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RITA (CD32), Fc gamma RIIB (CD32), Fc gamma RIIIA (CD16), and/or Fc gamma RIBB (CD16). The polynucleotide sequence and amino acid sequence of Fc gamma RI are shown in SEQ ID NOs: 80 (NM 000566.3) and 74 (NP 000557.1), respectively; the polynucleotide sequence and amino acid sequence of Fc gamma RIIA are shown in SEQ ID NOs: 81 (BC020823.1) and 75 (AAH20823.1), respectively; the polynucleotide sequence and amino acid sequence of Fc gamma RIIB are shown in SEQ ID NOs: 82 (BC146678.1) and 76 (AAI46679.1), respectively; the polynucleotide sequence and amino acid sequence of Fc gamma RIIIA are shown in SEQ ID NOs: 83 (BC033678.1) and 77 (AAH33678.1), respectively; and the polynucleotide sequence and amino acid sequence of Fc gamma RIIIB are shown in SEQ ID NOs: 84 (BC128562.1) and 78 (AAI28563.1), respectively (RefSeq accession number is shown in each parentheses). Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIM, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control antigen-binding molecule or antibody.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 62 (A is added to the N terminus of RefSeq accession number AAC82527.1), 63 (A is added to the N terminus of RefSeq accession number AAB59393.1), 64 (A is added to the N terminus of RefSeq accession number CAA27268.1), and 65 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fc domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:

(a) L234F, L235E, P331S;

(b) C226S, C229S, P238S;

(c) C226S, C229S; or

(d) C226S, C229S, E233P, L234V, L235A;

as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:

(e) H268Q, V309L, A330S, and P331S;

(f) V234A;

(g) G237A;

(h) V234A and G237A;

(i) A235E and G237A; or

(j) V234A, A235E, and G237A.

Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:

(k) F241A;

(l) D265A; or

(m) V264A.

Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:

(n) L235A, G237A, and E318A;

(o) L235E; or

(p) F234A and L235A.

Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

### Antigen-Binding Domains Bind to CLDN6

The phrase “an antigen-binding domain binds to CLDN6” or “an anti-CLDN6 antigen-binding domain” as used herein refers to an antigen-binding domain that specifically binds to the above-mentioned CLDN6 protein, or the whole or a portion of a partial peptide of the CLDN6 protein.

In certain embodiments, the antigen-binding domain binds to CLDN6 is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv₂ (scFv₂)”, “Fab”, “F(ab′)₂”, etc. In specific embodiments, the antigen-binding domain binds to CLDN6 is a domain comprising an antibody variable fragment. Domains comprising an antibody variable fragment may be provided from variable domains of one or a plurality of antibodies.

In certain embodiments, the antigen-binding domain binds to CLDN6 comprises the heavy-chain variable region and light-chain variable region of an anti-CLDN6 antibody. In certain embodiments, the antigen-binding domain binds to CLDN6 is a domain comprising a Fab structure.

Preferably, the anti-CLDN6 antibody comprises an H chain comprising the amino acid sequence (H-chain variable region) of any one of SEQ ID NOs: 7, 15, 23 or 31, and an L chain comprising the amino acid sequence (L-chain variable region) of any one of SEQ ID NOs: 8, 16, 24 or 32, respectively.

In some embodiments, the anti-CLDN6 antigen-binding domain binds specifically to the first extracellular domain of CLDN6 (amino acids 29-81 of SEQ ID NO: 44 or 45) or the second extracellular domain of CLDN6 (amino acids 138-159 of SEQ ID NO: 44 or 45). In some embodiments, the anti-CLDN6 antigen-binding domain binds specifically to an epitope within the first extracellular domain of CLDN6 (amino acids 29-81 of SEQ ID NO: 44 or 45) or the second extracellular domain of CLDN6 (amino acids 138-159 of SEQ ID NO: 44 or 45). In some embodiments, the anti-CLDN6 antigen-binding domain does not substantially binds to human CLDN9, preferably human CLDN9 expressed on the surface of eukaryotic cells; or does not substantially binds to human CLDN3, preferably human CLDN3 expressed on the surface of eukaryotic cells; or does not substantially binds to human CLDN4, preferably human CLDN4 expressed on the surface of eukaryotic cells. In some embodiments, the anti-CLDN6 antigen-binding domain does not substantially binds to a CLDN6 mutant as defined in SEQ ID NO: 53, preferably the CLDN6 mutant expressed on the surface of eukaryotic cells. In some embodiments, the anti-CLDN6 antigen-binding domain binds to the CLDN6 protein expressed on the surface of eukaryotic cells. In some embodiments, the anti-CLDN6 antigen-binding domain binds to the CLDN6 protein expressed on the surface of cancer cells.

In specific embodiments, the antigen-binding domain binds to CLDN6 comprises any one of the antibody variable fragments shown in Table 1 below.

TABLE 1
Sequences of HVRs in an antigen-binding domain binds to CLDN6

| Antibody | | | | | | |
|---|---|---|---|---|---|---|
| variable | SEQ ID NOs: | | | | | |
| fragment | HVR-H1 | HVR-H2 | HVR-H3 | HVR-L1 | HVR-L2 | HVR-L3 |
| 1 | 1 | 2 | 3 | 4 | 5 | 6 |
| 2 | 0 | 10 | 11 | 12 | 13 | 14 |
| 3 | 17 | 18 | 14 | 20 | 21 | 22 |
| 4 | 25 | 26 | 27 | 28 | 29 | 30 |

In specific embodiments, the antigen-binding domain binds to CLDN6 is a domain that comprises an antibody variable fragment that competes for binding to human CLDN6 with any one of the antibody variable fragments shown in Table 1. In specific embodiments, the antigen-binding domain binds to CLDN6 is a domain that comprises an antibody variable fragment that binds to the same epitope within human CLDN6 as any one of the antibody variable fragments shown in Table 1.

Alternatively, the antigen-binding domain binds to CLDN6 comprises an antibody variable fragment that competes for binding to human CLDN6 with any one of the above-mentioned antibody variable fragments. Alternatively, the antigen-binding domain binds to CLDN6 comprises an antibody variable fragment that binds to the same epitope to which any one of the above-mentioned antibody variable fragments binds on human CLDN6.

### Antigen-Binding Domains Bind to T Cell Receptor Complex

The phrase “an antigen-binding domain binds to T cell receptor complex” or “an anti-T cell receptor complex antigen-binding domain” as used herein refers to an antigen-binding domain that specifically binds to the whole or a portion of a partial peptide of a T cell receptor complex. The T cell receptor complex may be a T cell receptor itself, or an adaptor molecule constituting a T cell receptor complex along with a T cell receptor. CD3 is suitable as an adaptor molecule.

In certain embodiments, the antigen-binding domain binds to T cell receptor complex-binding activity is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv2 (scFv2)”, “Fab”, “F(ab′)2”, etc. In specific embodiments, the antigen-binding domain binds to T cell receptor complex is a domain comprising an antibody variable fragment. Domains comprising an antibody variable fragment may be provided from variable domains of one or a plurality of antibodies.

In certain embodiments, the antigen-binding domain binds to T cell receptor complex-comprises the heavy-chain variable region and light-chain variable region of an anti-T cell receptor complex antibody. In certain embodiments, the antigen-binding domain binds to T cell receptor complex is a domain comprising a Fab structure.

### Antigen-Binding Domains Bind to T Cell Receptor

The phrase “an antigen-binding domain binds to T cell receptor” or “an anti-T cell receptor antigen-binding domain” as used herein refers to an antigen-binding domain that specifically binds to the whole or a portion of a partial peptide of a T cell receptor. The portion of a T cell receptor to which the antigen-binding domain binds may be a variable region of the T cell receptor or a constant region of the T cell receptor; however, an epitope present in the constant region is preferred. Examples of the constant region sequence include the T cell receptor alpha chain of RefSeq Accession No. CAA26636.1 (SEQ ID NO: 54), the T cell receptor beta chain of RefSeq Accession No. C25777 (SEQ ID NO: 55), the T cell receptor gamma 1 chain of RefSeq Accession No. A26659 (SEQ ID NO: 56), the T cell receptor gamma 2 chain of RefSeq Accession No. AAB63312.1 (SEQ ID NO: 57), and the T cell receptor delta chain of RefSeq Accession No. AAA61033.1 (SEQ ID NO: 58).

In certain embodiments, the antigen-binding domain binds to T cell receptor is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv2 (scFv2)”, “Fab”, “F(ab′)2”, etc. In specific embodiments, the antigen-binding domain binds to T cell receptor is a domain comprising an antibody variable fragment. Domains comprising an antibody variable fragment may be provided from variable domains of one or a plurality of antibodies.

In certain embodiments, the antigen-binding domain binds to T cell receptor comprises the heavy-chain variable region and light-chain variable region of an anti-T cell receptor antibody. In certain embodiments, the antigen-binding binds to T cell receptor is a domain comprising a Fab structure.

### Antigen-Binding Domains Bind to CD3

The phrase “an antigen-binding domain binds to CD3” or “an anti-CD3 antigen-binding domain” as used herein refers to an antigen-binding domain that specifically binds to the whole or a portion of a partial peptide of CD3. The antigen-binding domain binds to CD3 may be any epitope-binding domain as long as the epitope exists in the gamma-chain, delta-chain, or epsilon-chain sequence that constitutes human CD3. Regarding the structure of the gamma chain, delta chain, or epsilon chain constituting CD3, their polynucleotide sequences are disclosed in RefSeq Accession NOs. NM_000073.2, NM_000732.4 and NM_000733.3, and their polypeptide sequences are shown in SEQ ID NOs: 59 (NP_000064.1), 60 (NP_000723.1), and 61 (NP_000724.1), wherein the RefSeq accession numbers are shown in parentheses.

In certain embodiments, the antigen-binding domain binds to CD3 is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc. In specific embodiments, the antigen-binding domain binds to CD3 is a domain comprising an antibody variable fragment. Domains comprising an antibody variable fragment may be provided from variable domains of one or a plurality of antibodies.

In certain embodiments, the antigen-binding domain binds to CD3 comprises the heavy-chain variable region and light-chain variable region of an anti-CD3 antibody. In certain embodiments, the antigen-binding domain binds to CD3 is a domain comprising a Fab structure.

The antigen-binding domains bind to CD3-binding activity of the present invention may bind to any epitope, as long as the epitope is located within the gamma chain, delta chain, or epsilon chain sequence forming human CD3. In the present invention, preferred antigen-binding domains bind to CD3 include those comprising a CD3 antibody light-chain variable region (VL) and a CD3 antibody heavy-chain variable region (VH), which bind to an epitope in the extracellular domain of the epsilon chain of a human CD3 complex. Such preferred antigen-binding domains bind to CD3 include those comprising a CD3 antibody light-chain variable region (VL) and a CD3 antibody heavy-chain variable region (VH) of the OKT3 antibody (Proc. Natl. Acad. Sci. USA (1980) 77, 4914-4917) or various known CD3 antibodies such as an antibody with the light-chain variable region (VL) of NCBI Accession No. AAB24132 and the heavy-chain variable region (VH) of NCBI Accession No. AAB24133 (Int. J. Cancer Suppl. 7, 45-50 (1992)). Furthermore, such appropriate antigen-binding domains bind to CD3 include those derived from a CD3 antibody with desired characteristics, which are obtained by immunizing a desired animal with the gamma chain, delta chain, or epsilon chain forming human CD3 by the above-described methods. Appropriate anti-CD3 antibodies from which an antigen-binding domain binds to CD3 is derived include human antibodies and antibodies appropriately humanized as described above.

### Multispecific Antigen-Binding Molecules

“Multispecific antigen-binding molecules” refers to antigen-binding molecules that bind specifically to more than one antigen. In a favorable embodiment, multispecific antigen-binding molecules of the present invention comprise two or more antigen-binding domains, and different antigen-binding domains bind specifically to different antigens.

The multispecific antigen-binding molecule of the present invention comprises a first antigen-binding domain binds to CLDN6, and a second antigen-binding domain binds to T cell receptor complex. The combinations of an antigen-binding domain binds to CLDN6 selected from those described in “Antigen-binding domains bind to CLDN6” above and an antigen-binding domain binds to T cell receptor complex selected from those described in “Antigen-binding domains bind to T-cell receptor complex” to “Antigen-binding domains binds to CD3” above can be used.

For example, the first antigen-binding domain is a domain comprising antibody heavy-chain and light-chain variable regions, and/or the second antigen-binding domain is a domain comprising antibody heavy-chain and light-chain variable regions. Alternatively, the first antigen-binding domain is a domain comprising an antibody variable fragment, and/or the second antigen-binding domain is a domain comprising an antibody variable fragment. Alternatively, the first antigen-binding domain is a domain comprising a Fab structure, and/or the second antigen-binding domain is a domain comprising a Fab structure.

In certain embodiments, the present invention provides a multispecific antigen-binding molecule comprising a first antigen-binding domain that comprises an antibody variable fragment that binds to CLDN6, and a second antigen-binding domain that comprises an antibody variable fragment that binds to T cell receptor complex. In certain embodiments, the present invention provides bispecific antigen-binding molecules that comprise a first antigen-binding domain binds to CLDN6, a second antigen-binding domain binds to T cell receptor complex, and a third domain comprising an Fc region that has a reduced Fc gamma receptor-binding activity. The Fc region may have a reduced Fc gamma receptor-binding activity compared with the Fc domain of an IgG1, IgG2, IgG3, or IgG4 antibody. In an embodiment, the Fc region is an Fc region with an amino acid mutation at any of the Fc region-constituting amino acids of SEQ ID NOs: 62 to 65 (IgG1 to IgG4).

In certain embodiments, the present invention provides bispecific antibodies that comprise a first antibody variable fragment that binds to human CLDN6, and a second antibody variable fragment that binds to CD3. In certain embodiments, the present invention provides bispecific antibodies that comprise a first antibody variable fragment binds to human CLDN6, a second antibody variable fragment binds to CD3, and an Fc region that has a reduced Fc gamma receptor-binding activity. In certain embodiments, the present invention provides bispecific antibodies that comprise a first antibody variable fragment binds to human CLDN6, a second antibody variable fragment that binds to CD3 eplison chain, and an Fc region that has a reduced Fc gamma receptor-binding activity compared with naturally occurring IgG Fc regions.

Examples of a preferred embodiment of the “multispecific antigen-binding molecule” of the present invention include multispecific antibodies. When an Fc region with reduced Fc gamma receptor-binding activity is used as the multispecific antibody Fc region, an Fc region derived from the multispecific antibody may be used appropriately. Bispecific antibodies are particularly preferred as the multispecific antibodies of the present invention. In this case, a bispecific antibody is an antibody having two different specificities. IgG-type bispecific antibodies can be secreted from a hybrid hybridoma (quadroma) produced by fusing two types of hybridomas that produce IgG antibodies (Milstein et al., Nature (1983) 305, 537-540).

Furthermore, IgG-type bispecific antibodies are secreted by introducing the genes of L chains and H chains constituting the two types of IgGs of interest, i.e., a total of four genes, into cells, and co-expressing them. However, the number of combinations of H and L chains of IgG that can be produced by these methods is theoretically ten combinations. Accordingly, it is difficult to purify an IgG comprising the desired combination of H and L chains from ten types of IgGs. Furthermore, theoretically, the amount of secretion of the IgG having the desired combination will decrease remarkably, and therefore large-scale culturing will be necessary, and production costs will increase further.

Therefore, techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:

(a) glutamic acid (E) and aspartic acid (D); and

(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

Other known techniques can also be used for the association of multispecific antibodies of the present invention. Fc region-containing polypeptides comprising different amino acids can be efficiently associated with each other by substituting an amino acid side chain present in one of the H-chain Fc regions of the antibody with a larger side chain (knob), and substituting an amino acid side chain present in the corresponding Fc region of the other H chain with a smaller side chain (hole) to allow placement of the knob within the hole (WO1996/027011; Ridgway J B et al., Protein Engineering (1996) 9, 617-621; Merchant A. M. et al. Nature Biotechnology (1998) 16, 677-681; and US20130336973).

In addition, other known techniques can also be used for formation of multispecific antibodies of the present invention. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing bispecific antibodies composed of two types of light chains and one type of heavy chain as described in WO2012/023053; technologies for producing bispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody of the present invention can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

A plurality, such as two or more, of these technologies can be used in combination. Furthermore, these technologies can be appropriately and separately applied to the two H chains to be associated. Furthermore, these techniques can be used in combination with the above-mentioned Fc region which has reduced binding activity to an Fc gamma receptor. Furthermore, an antigen-binding molecule of the present invention may be a molecule produced separately so that it has the same amino acid sequence, based on the antigen-binding molecule subjected to the above-described modifications.

Preferably, the antigen-binding molecule of the present invention may comprise a first antigen-binding domain binds to CLDN6, and a second antigen-binding domain binds to T cell receptor complex. In an embodiment, the second antigen-binding domain binds to T cell receptor. In another embodiment, the second antigen-binding domain binds to CD3 epsilon chain. In an embodiment, the first antigen-binding domain binds to human CLDN6. In a further embodiment, the first antigen-binding domain binds to CLDN6 on the surface of a eukaryotic cell. In an embodiment, the first antigen-binding domain binds to human CLDN6 on the surface of a eukaryotic cell including cancer cell.

The phrase “anti-CLDN6 arm” in this specification refers to the antibody heavy chain and antibody light chain which binds to CLDN6 in a bispecific antibody. The phrase “anti-CD3 arm” in this specification refers to the antibody heavy chain and antibody light chain which binds to CD3 in a bispecific antibody.

Preferably, the antigen-binding molecule of the present invention may have cellular cytotoxicity (also referred to as “cytotoxicity”). In an embodiment, the cellular cytotoxicity is T cell-dependent cellular cytotoxicity (TDCC). In another embodiment, the cytotoxicity is a cellular cytotoxicity towards cells expressing CLDN6 on their surfaces. The CLDN6-expressing cells may be cancer cells.

In a preferred aspect, an antibody (or antigen-binding molecule) of the present invention has cytotoxicity (or cellular cytotoxicity), or preferably T cell-dependent cellular cytotoxicity (TDCC) against CLDN6-expressing cells such as cancer cells. CLDN6 may be expressed on the surface of such cells. The (cellular) cytotoxicity or TDCC of an antibody (or antigen-binding molecule) of the present invention can be evaluated by any suitable method known in the art. For example, TDCC can be measured by lactate dehydrogenase (LDH) release assay as described in Example 3. In this assay, target cells (e.g. CLDN6-expressing cells) are incubated with T cells (e.g. PBMCs) in the presence of a test antibody, and the activity of LDH that has been released from target cells killed by T cells is measured using a suitable reagent. Typically, the cytotoxic activity is calculated as a percentage of the LDH activity resulting from the incubation with the antibody relative to the LDH activity resulting from 100% killing of target cells (e.g. lysed by treatment with Triton-X). If the cytotoxic activity calculated as mentioned above is higher, the test antibody is determined to have higher TDCC. Additionally or alternatively, for example, TDCC can also be measured by real-time cell growth inhibition assay as described in Example 5. In this assay, target cells (e.g. CLDN6-expressing cells) are incubated with T cells (e.g. PBMCs) in the presence of a test antibody on a 96-well plate, and the growth of the target cells is monitored by methods known in the art, for example, by using a suitable analyzing instrument (e.g. xCELLigence Real-Time Cell Analyzer). The rate of cell growth inhibition (CGI: %) is determined from the cell index value according to the formulation given as CGI (%)=100−(CIAb×100/CINoAb). “CIAb” represents the cell index value of wells with the antibody on a specific experimental time and “CINoAb” represents the average cell index value of wells without the antibody. If the CGI rate of the antibody is high, i.e., has a significantly positive value, it can be said that the antibody has TDCC activity and is more preferable in the present invention.

In a preferred aspect, an antibody (or antigen-binding molecule) of the present invention has T cell activation activity. T cell activation can be assayed by methods known in the art, such as a method using an engineered T cell line that expresses a reporter gene (e.g. luciferase) in response to its activation (e.g. Jurkat/NFAT-RE Reporter Cell Line (T Cell Activation Bioassay, Promega)). In this method, target cells (e.g. CLDN6-expressing cells) are cultured with T cells in the presence of a test antibody, and then the level or activity of the expression product of the reporter gene is measured by appropriate methods as an index of T cell activation. When the reporter gene is a luciferase gene, luminescence arising from reaction between luciferase and its substrate may be measured as an index of T cell activation. If T cell activation measured as described above is higher, the test antibody is determined to have higher T cell activation activity.

### Cancer

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### Tumor

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Ovarian Cancer

“Ovarian cancer” refers to a heterogeneous group of malignant tumors derived from the ovary. Approximately 90% of malignant ovarian tumors are epithelial in origin; the remainder are germ cell and stromal tumors. Epithelial ovarian tumors are classified into the following histological subtypes: serous adenocarcinomas (constituting about 50% of epithelial ovarian tumors); endometrioid adenocarcinomas (˜20%); mucinous adenocarcinomas (˜10%); clear cell carcinomas (˜5-10%), Brenner (transitional cell) tumors (relatively uncommon). The prognosis for ovarian cancer, which is the sixth most common cancer in women, is usually poor, with five year survival rates ranging from 5-30%. For reviews of ovarian cancer, see Fox et al. (2002) “Pathology of epithelial ovarian cancer,” in Ovarian Cancer ch. 9 (Jacobs et al., eds., Oxford University Press, New York); Morin et al. (2001) “Ovarian Cancer,” in Encyclopedic Reference of Cancer, pp. 654-656 (Schwab, ed., Springer-Verlag, New York). The present invention contemplates methods of diagnosing or treating any of the epithelial ovarian tumor subtypes described above, and in particular, the serous adenocarcinoma subtype.

### Gastric Cancer

The term “gastric cancer”, or “gastric tumor”, or “stomach tumor”, or “stomach cancer” refers to any tumor or cancer of the stomach, including, e.g., adenocarcinomas (such as diffuse type and intestinal type), and less prevalent forms such as lymphomas, leiomyosarcomas, and squamous cell carcinomas.

### Pharmaceutical Formulation

The term “pharmaceutical formulation” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

In one aspect, the invention is based, in part, on multispecific antigen-binding molecules that comprises a first domain comprising a first antibody variable region that binds to CLDN6, and a second domain comprising a second antibody variable region that binds to T-cell receptor complex, and use thereof. Antigen-binding molecules and antibodies of the invention are useful, e.g., for the diagnosis or treatment of cancer, especially ovarian tumor, non-small cell lung cancer, gastric cancer, and liver cancer.

### Pharmaceutical Composition

A pharmaceutical composition of the present invention, a therapeutic agent for inducing cellular cytotoxicity, a cell growth-suppressing agent, or an anticancer agent of the present invention may be formulated with different types of multispecific antigen-binding molecules, if needed. For example, the cytotoxic action against cells expressing an antigen can be enhanced by a cocktail of multiple multispecific antigen-binding molecules of the present invention.

If necessary, the multispecific antigen-binding molecules of the present invention may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for preparing agents as sustained-release agents are known, and these can be applied to the multispecific antigen-binding molecules of the present invention (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

The pharmaceutical compositions, cell growth-suppressing agents, or anticancer agents of the present invention may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present invention is not limited to these doses.

The pharmaceutical compositions of the present invention can be formulated according to conventional methods (for example, Remington's Pharmaceutical Science, latest edition, Mark Publishing Company, Easton, U.S.A.), and may also contain pharmaceutically acceptable carriers and additives. Examples include, but are not limited to, surfactants, excipients, coloring agents, flavoring agents, preservatives, stabilizers, buffers, suspension agents, isotonic agents, binders, disintegrants, lubricants, fluidity promoting agents, and corrigents, and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such.

Preferably, a pharmaceutical composition of the present invention comprises a multispecific antigen-binding molecule of the invention. In one aspect, the composition is a pharmaceutical composition for use in inducing cellular cytotoxicity. In another aspect, the composition is a pharmaceutical composition for use in treating or preventing cancer. Preferably, the cancer is colorectal cancer or gastric cancer. The pharmaceutical composition of the present invention can be used for treating or preventing cancer. Thus, the present invention provides a method for treating or preventing cancer, in which the multispecific antigen-binding molecule of the present invention is administered to a patient in need thereof

The present invention also provides methods for damaging cells expressing CLDN6 or for suppressing the cell growth by contacting the cells expressing CLDN6 with a multispecific antigen-binding molecule of the present invention that binds to CLDN6. Monoclonal antibodies that bind to CLDN6 are described above as a multispecific antigen-binding molecule of the present invention, which is included in the therapeutic agents for inducing cellular cytotoxicity, cell growth-suppressing agents, and anticancer agents of the present invention. Cells to which a multispecific antigen-binding molecule of the present invention binds are not particularly limited, as long as they express CLDN6. Specifically, in the present invention, the preferred cancer antigen-expressing cells include ovary cancer cells, prostate cancer cells, breast cancer cells, uterine cancer cells, liver cancer cells, lung cancer cells, pancreatic cancer cells, stomach cancer cells, urinary bladder cancer cells, and colon cancer cells.

In the present invention, “contact” can be carried out, for example, by adding a multispecific antigen-binding molecule of the present invention to culture media of cells expressing CLDN6 cultured in vitro. In this case, a multispecific antigen-binding molecule to be added can be used in an appropriate form, such as a solution or solid prepared by lyophilization or the like. When the multispecific antigen-binding molecule of the present invention is added as an aqueous solution, the solution may be a pure aqueous solution containing the multispecific antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In another embodiment of the present invention, “contact” can also be carried out by administration to nonhuman animals transplanted with CLDN6-expressing cells in vivo or to animals having cancer cells expressing CLDN6 endogenously. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, an appropriate administration method can be selected according to the age and symptoms of an animal subject. When the multispecific antigen-binding molecule is administered as an aqueous solution, the solution may be a pure aqueous solution containing the multispecific antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of a multispecific antigen-binding molecule of the present invention is not limited to these examples.

The methods described below are preferably used as a method for assessing or determining cellular cytotoxicity caused by contacting a multispecific antigen-binding molecule of the present invention with CLDN6-expressing cells to which the antigen-binding domain forming the multispecific antigen-binding molecules of the present invention binds. The methods for assessing or determining the cytotoxic activity in vitro include methods for determining the activity of cytotoxic T-cells or the like. Whether a multispecific antigen-binding molecule of the present invention has the activity of inducing T-cell mediated cellular cytotoxicity can be determined by known methods (see, for example, Current protocols in Immunology, Chapter 7. Immunologic studies in humans, Editor, John E, Coligan et al., John Wiley & Sons, Inc., (1993)). In the cytotoxicity assay, a multispecific antigen-binding molecule whose antigen-binding domain binds to an antigen different from CLDN6 and which is not expressed in the cells is used as a control multispecific antigen-binding molecule. The control multispecific antigen-binding molecule is assayed in the same manner. Then, the activity is assessed by testing whether a multispecific antigen-binding molecule of the present invention exhibits a stronger cytotoxic activity than that of a control multispecific antigen-binding molecule.

Meanwhile, the in vivo anti-tumor efficacy is assessed or determined, for example, by the following procedure. Cells expressing the antigen to which the antigen-binding domain forming a multispecific antigen-binding molecule of the present invention binds are transplanted intracutaneously or subcutaneously to a nonhuman animal subject. Then, from the day of transplantation or thereafter, a test multispecific antigen-binding molecule is administered into vein or peritoneal cavity every day or at intervals of several days. The tumor size is measured over time. Difference in the change of tumor size can be defined as the cytotoxic activity. As in an in vitro assay, a control multispecific antigen-binding molecule is administered. The multispecific antigen-binding molecule of the present invention can be judged to have cytotoxic activity when the tumor size is smaller in the group administered with the multispecific antigen-binding molecule of the present invention than in the group administered with the control multispecific antigen-binding molecule.

An MTT method and measurement of isotope-labeled thymidine uptake into cells are preferably used to assess or determine the effect of contact with a multispecific antigen-binding molecule of the present invention to suppress the growth of cells expressing an antigen to which the antigen-binding domain forming the multispecific antigen-binding molecule binds. Meanwhile, the same methods described above for assessing or determining the in vivo cytotoxic activity can be used preferably to assess or determine the activity of suppressing cell growth in vivo.

The present invention also provides kits for use in a method of the present invention, which contain a multispecific antigen-binding molecule of the present invention or a multispecific antigen-binding molecule produced by a method of the present invention. The kits may be packaged with an additional pharmaceutically acceptable carrier or medium, or instruction manual describing how to use the kits, etc.

In addition, the present invention relates to multispecific antigen-binding molecules of the present invention or multispecific antigen-binding molecules produced by a method of the present invention for use in a method of the present invention.

All documents cited herein are incorporated herein by reference.

### EXAMPLES

The following are examples of methods and compositions of the invention. It is understood that various other embodiments may be practiced, given the general description provided above.

### Example 1. Generation of CLDN6-Targeted T-Cell Redirecting Bispecific Antibodies (CLDN6-TRAB)

T cell redirecting bispecific antibodies which direct against CLDN6 were generated. To generate CLDN6-targeted T-cell redirecting bispecific antibodies (hereinafter referred as CLDN6-TRABs), four anti-CLDN6 bivalent antibodies (CH01, CH02, CH03, CH04) were used for CLDN6-binding arm, and one anti-CD3 bivalent antibody were used for CD3-binding arm of CLDN6-TRAB. cDNAs encoding the antibodies including variable region and human IgG1 constant region were synthesized and cloned into the standard mammalian expression vector. Each of the anti-CLDN6 bivalent antibodies and the anti-CD3 bivalent antibody was transiently transfected and expressed using an Expi293 Expression system (Thermo Fisher Scientific). Culture supernatants were harvested, and antibodies were purified from the supernatants using MabSelect SuRe pcc affinity chromatography (GE Healthcare) and subsequent gel permeate chromatography using Superdex200 (GE Healthcare). To generate the CLDN6-TRABs, purified four anti-CLDN6 bivalent antibodies CH01, CH02, CH03, and CH04 were subjected to Fab arm exchanging technology (as described in WO2015/046467) with the anti-CD3 bivalent antibody mentioned above. Four CLDN6-TRABs were generated and named CH01/CD3, CH02/CD3, CH03/CD3, and CH04/CD3 respectively. Summary and sequences of the CLDN6-TRABs are shown in Table 2.

TABLE 2
Summary and sequences of CLDN6-TRABs

| Antibody name | | | CH01/CD3 | CH02/CD3 | CH03/CD3 | CH04/CD3 |
|---|---|---|---|---|---|---|
| anti-CLDN6 arm originated from | | | CH01 | CH02 | CH03 | CH04 |
| anti-CD3 arm originated from | | | CD3-1 | | | |
| SEQ ID NOs: | anti-CLDN6 | HCDR1 | 1 | 9 | 17 | 25 |
| | arm | HCDR2 | 2 | 10 | 18 | 26 |
| | | HCDR3 | 3 | 11 | 19 | 27 |
| | | Heavy chain | 7 | 15 | 23 | 31 |
| | | variable region | | | | |

| Heavy chain | 41 | | | |
|---|---|---|---|---|
| constant region | | | | |
| LCDR1 | 4 | 12 | 20 | 28 |
| LCDR2 | 5 | 13 | 21 | 29 |
| LCDR3 | 6 | 14 | 22 | 30 |
| Light chain | 8 | 16 | 24 | 32 |
| variable region | | | | |

| | | |
|---|---|---|
| | Light chain | 43 |
| | constant region | |
| anti-CD3 | HCDR1 | 33 |
| arm | HCDR2 | 34 |
| | HCDR3 | 35 |
| | Heavy chain | 39 |
| | variable region | |
| | Heavy chain | 42 |
| | constant region | |
| | LCDR1 | 36 |
| | LCDR2 | 37 |
| | LCDR3 | 38 |
| | Light chain | 40 |
| | variable region | |
| | Light chain | 43 |
| | constant region | |

### Example 2. Binding Activity and Specificity of CLDN6-TRABs

To evaluate the binding activity and specificity of the generated CLDN6-TRABs against CLDN6 and other Claudin family antigens, FACS analysis was conducted as follows. Ba/F3 transfectants stably express a kind of protein selected from human CLDN3 (hCLDN3), human CLDN4 (hCLDN4), human CLDN6 (hCLDN6), human CLDN9 (hCLDN9), mouse CLDN3 (mCLDN3), mouse CLDN4 (mCLDN4) and mouse CLDN6 (mCLDN6), were generated using similar method described in WO2008/087978, and named BaF3_hCLDN3, BaF3_hCLDN4, BaF3_hCLDN6, BaF3_hCLDN9, BaF3 mCLDN3, BaF3 mCLDN4, BaF3 mCLDN6, respectively. Each of the CLDN6-TRABs (CH01/CD3, CH02/CD3, CH03/CD3, CH04/CD3) was incubated with the Ba/F3 transfectants at an antibody concentration of 20 micro g/ml in FACS buffer (0.5% BSA containing autoMACS Rinsing Solution, Miltenyl Biotec). Binding activities of each CLDN6-TRAB to the Claudin family proteins (hCLDN3, hCLDN4, hCLDN6, hCLDN9, mCLDN3, mCLDN4, mCLDN6) were examined by incubation of FITC-conjugated goat anti human IgG (BeckmanCoulter), represented as Geo-mean value. Sample processing was conducted on FACSVerse (BD).

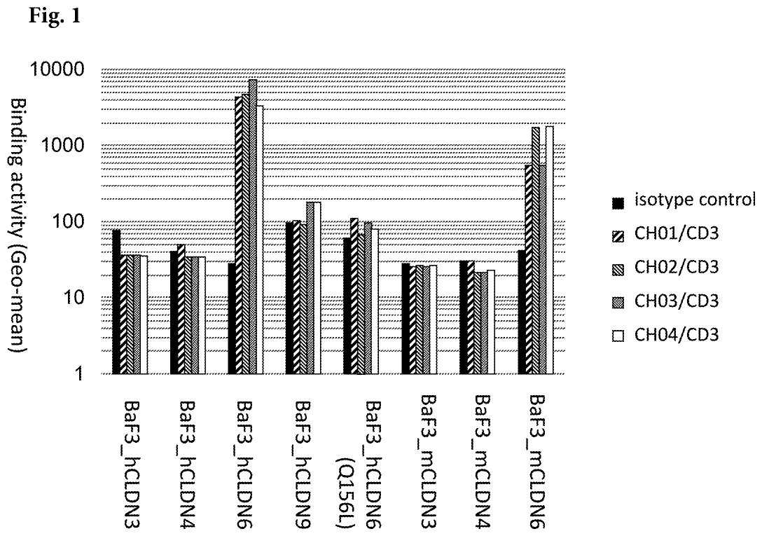

As shown in FIG. 1 and Table 3, all the CLDN6-TRABs bind to the human or mouse CLDN6 expressed on the cell surface of Ba/F3. Binding of the tested antibodies against mouse CLDN9 is predictable from their binding against human CLDN9, because the amino acid sequences of the extracellular domains of mouse CLDN9 are completely the same with the ones of human CLDN9 extracellular domains.

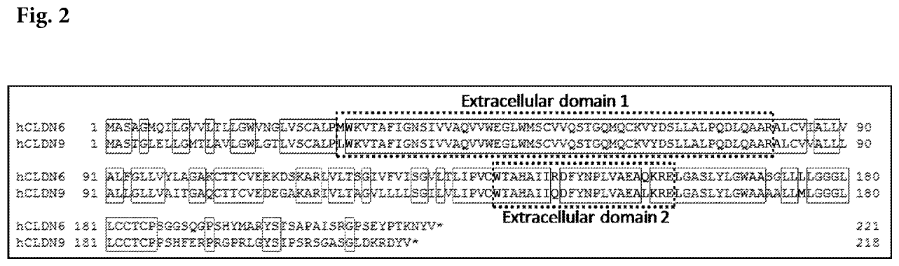

Claudin family proteins have two extracellular domains. The sequence of the first extracellular domain in human CLDN9 is almost the same compared with the first extracellular domain of human CLDN6. For the second extracellular domain of human CLDN9, there are only two amino acids different from the ones at their corresponding positions in human CLDN6 (FIG. 2). The glutamine at position 156 of human Claudin 6 (position 156 in the sequence shown in SEQ ID NO: 44 or 45) was substituted to leucine to make a human CLDN6 mutant comprising the same amino acid as human Claudin 9 at position 156. This human CLDN6 mutant was named as hCLDN6(Q156L) (SEQ ID NO: 53). Ba/F3 transfectant stably express hCLDN6(Q156L) was generated using similar method described in WO2008/087978. The established Ba/F3 transfectant was named BaF3_hCLDN6(Q156L). Binding activity of the generated CLDN6-TRABs against BaF3_hCLDN6(Q156L) was measured using the above mentioned FACS method and the results are shown in FIG. 1 and Table 3.

TABLE 3
Binding activity of various CLDN6-TRABs to Ba/F3
transfectants, shown by Geo-mean value of FACS.

| | | Antibody | | | | |
|---|---|---|---|---|---|---|
| | | isotype control | CH01/CD3 | CH02/CD3 | CH03/CD3 | CH04/CD3 |
| Cell | BaF3_hCLDH3 | 78 | 37 | 37 | 37 | 36 |
| | BaF3_hCLDH4 | 42 | 51 | 35 | 35 | 34 |
| | BaF3_hCLDN6 | 29 | 4476 | 4740 | 7701 | 3481 |
| | BaF3_hCLDH9 | 96 | 104 | 84 | 184 | 186 |
| | BaF3_hCLDN6(Q156L) | 62 | 114 | 69 | 98 | 80 |
| | BaF3_mCLDN3 | 29 | 26 | 27 | 26 | 27 |
| | BaF3_mCLDN4 | 31 | 31 | 22 | 22 | 23 |
| | BaF3_mCLDN6 | 43 | 550 | 1759 | 559 | 1790 |

### Example 3. In Vitro Assay of Cytotoxic Activity Using Lactate Dehydrogenase Release Assay

Cytotoxic activity of the CLDN6-TRABs was evaluated by lactate dehydrogenase (LDH) release assay.

Human ovarian cancer cell line OV-90 (ATCC) which expresses human CLDN6 was used as target cells.

50 mL of peripheral blood was collected from healthy adult volunteers by a syringe that had been previously injected with 500 micro L of 1,000 units/mL heparin solution (NovoNordisk). Peripheral blood equally divided into four equal parts by dilution with PBS (−) was injected with 15 mL of Ficoll-Paque PLUS and centrifuged in a Leucosep lymphocyte separation tube (Greiner Bio-One). After centrifuging the separation tube (2150 rpm for 10 minutes at room temperature), the peripheral blood mononuclear cell (hereinafter referred as PBMC) fraction layer was separated. After washing the PBMCs once with RPMI-1640 Medium (SIGMA) containing 10% FBS (called as 10% FBS/RPMI), the PBMCs were adjusted to 2×10⁶ cells/mL. These PBMCs were used as effector cell.

Target cells were detached from culture flask and seeded at 100 micro L/well containing 1×10⁴ cells on flat bottom clear 96-wells plate (Corning). 50 micro L of human PMBC solution (2×10⁵ cells) and 50 micro L of prepared antibody at a concentration selected from 0.004, 0.04, 0.4, 4, 40 nM were added into wells respectively. After overnight incubation at 37 degree C., plate was centrifuged and 100 micro L culture supernatants from each wells were transferred to new flat bottom clear 96-wells plate. Then 100 micro L of LDH detection reagent (Dye solution containing catalyst, TaKaRa) was added to each well, followed by 30 minutes incubation at room temperature. After adding 1N HCl (Wako Pure Chemical Industries) to stop the LDH enzyme reaction, absorbance at 490 nm and 620 nm was measured by EnVision (PerkinElmer Japan).

The rate of cytotoxic activity (%) was calculated from the difference between 490 nm and 620 nm absorbance according to following formula.

Cytotoxic activity (%)={(Ab_{Ab}−Abₛₚₒₙ)×100/(Ab_{TritonX}−Abₛₚₒₙ)}−{(Ab_{NoAb}−Abₛₚₒₙ)×100/(Ab_{TritonX}−Abₛₚₒₙ)}.

“Ab_{Ab}” represents the difference between 490 nm and 620 nm absorbance of wells contains the antibody, “Abₛₚₒₙ” represents the average absorbance value of the wells without antibody and effector cells, “Ab_{TritonX}” represents the average value of the wells which were added 20% Triton-X after overnight culture, and “Ab_{NoAb}” represents the average absorbance value of the wells without antibody. All the absorbance values were subtracted the average absorbance value of culture medium wells.

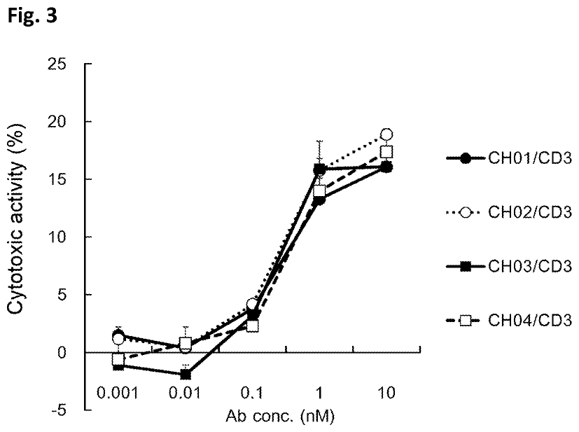

The results are shown in FIG. 3.

### Example 4. T Cell Activation Assay Using Jurkat Reporter Gene Assay

T cell activation activity of the CLDN6-TRABs was evaluated using Jurkat/NFAT-RE Reporter Cell Line (T Cell Activation Bioassay, Promega). Human cancer cell lines which endogenously express human claudin-6, including human lung adenocarcinoma cell lines ABC-1 (Health Science Research Resources Bank), human teratocarcinoma cell lines PA-1 (ATCC), human stomach choriocarcinoma cell lines SCH (Health Science Research Resources Bank), human hepatocellular carcinoma cell line HuH-7 (Health Science Research Resources Bank) and human ovarian cancer cell line OV-90 (ATCC), were used as target cells. In addition to these cancer cell lines, Ba/F3 transfectants stably express human CLDN6, CLDN9 or CLDN6(Q156L) (BaF3_hCLDN6, BaF3_hCLDN9, BaF3_hCLDN6(Q156L)) were also used as target cells. Assay was performed described as below. First, target cell was detached or corrected from culture flask and plated at 25 micro L/well (2×10⁴ cells) into white flat bottom 96-wells plate (Corning). Next, 1×10⁵ Jurkat/NFAT-RE Reporter Cell Line together with antibody at a concentration selected from 0.003, 0.03, 0.3, 3 and 30 nM were added at 25 micro L/well, respectively. After overnight culture at 37 degree C., Bio-Glo reagent (Promega) was added at 75 micro L/well followed by further incubation at room temperature for 10 minutes. Then, luminescence arising from activating Jurkat cells was measured by EnVision (PerkinElmer Japan). The luminescence ratio of each wells were calculated by comparing between the wells with and without antibody.

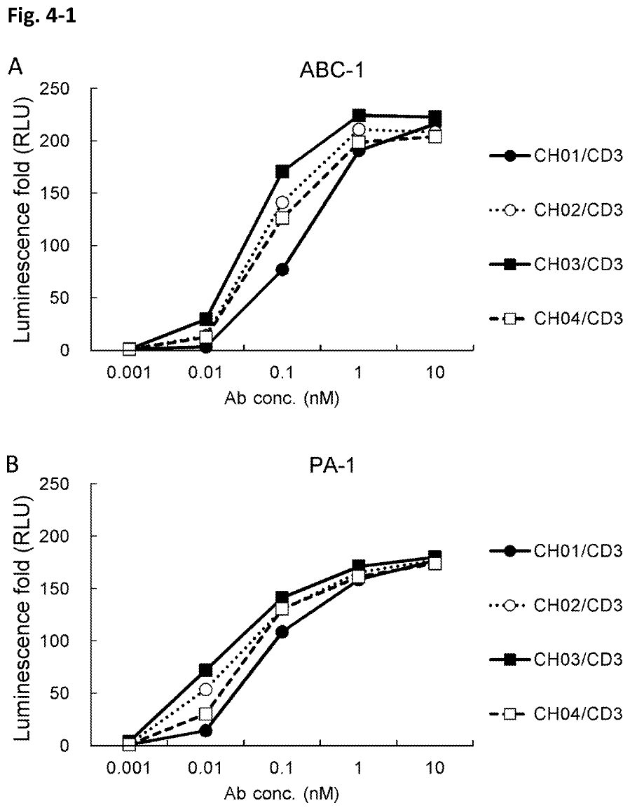

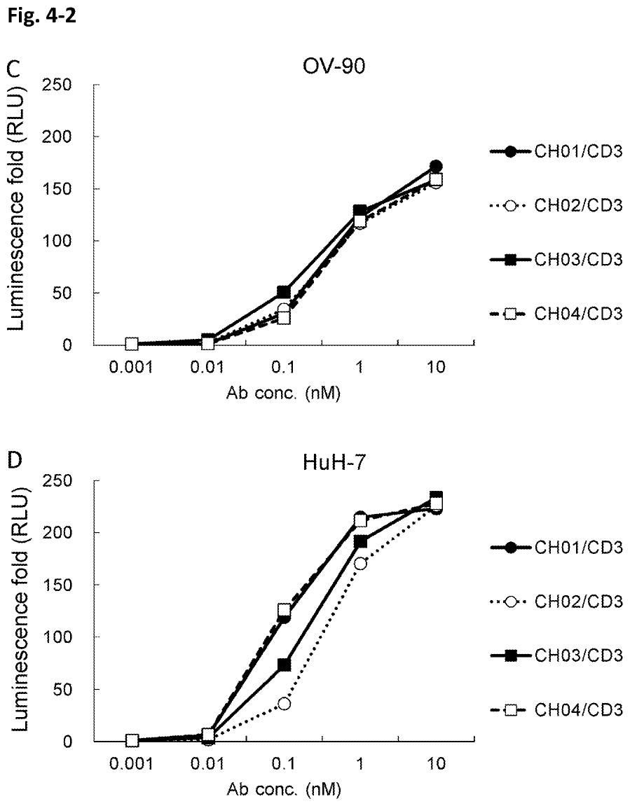

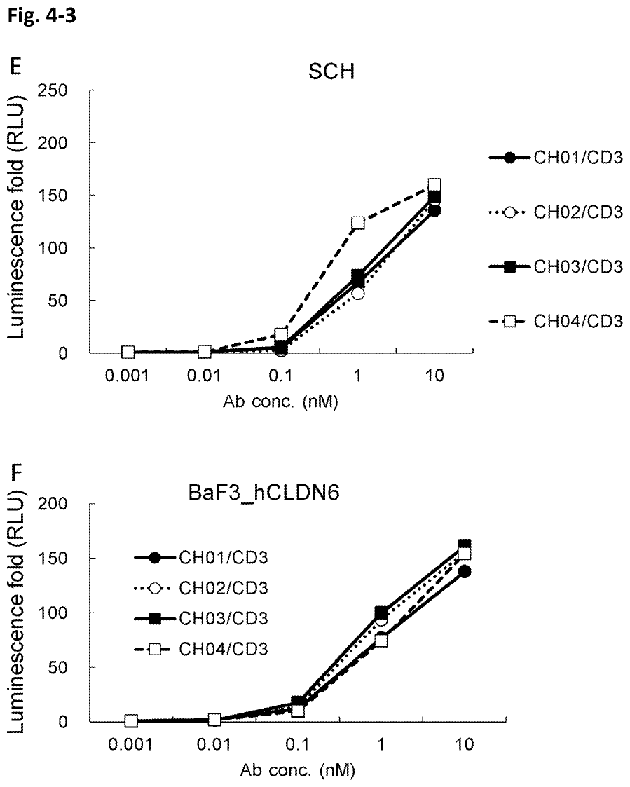

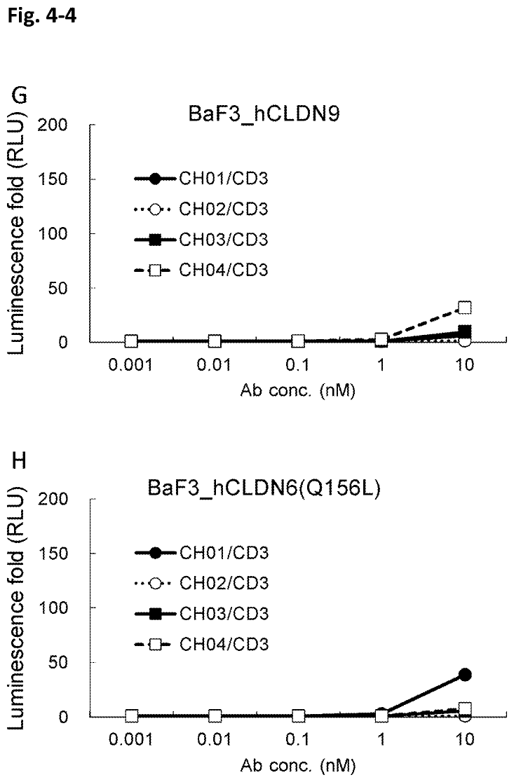

The results of T cell activation using CLDN6 expressing human cell lines (ABC-1, SCH, PA-1, HuH-7, and OV-90) as target cells are shown in FIG. 4A to FIG. 4E. The results using Ba/F3 transfectants (BaF3_hCLDN6, BaF3_hCLDN9 and BaF3_hCLDN6(Q156L)) as target cells are shown in FIG. 4F to FIG. 4H.

### Example 5. Real-Time Cell Growth Inhibition Assay (xCELLigence Assay)

The T-cell dependent growth inhibition mediated by the CLDN6-TRABs was assessed by cell proliferation assay using an xCELLigence RTCA MP instrument (ACEA Biosciences). CLDN6 expressing cancer cell lines HuH-7 and OV-90 were used as target cells. Expansion of T cells as effector cell was performed using human PBMC (AllCells) and T Cell Activation/Expansion Kit (Miltenyi Biotec). 1×10⁴ HuH-7 or OV-90 was plated on E-Plate 96 plate (Roche Diagnostics) at 100 micro L/well. After overnight culture, 2×10⁴ T cells together with antibody at a concentration selected from 0.004, 0.04, 0.4, 4 or 40 nM were added at 50 micro L/well, respectively. Cell growth was monitored every 15 min, using xCELLigence, for 72 hours during the incubation of the plates. The rate of cell growth inhibition (CGI: %) was determined from the cell index value according to the formulation given as CGI (%)=100−(CI_{Ab}×100/CI_{NoAb}). “CI_{Ab}” represents the cell index value of wells with antibody on a specific experimental time and “CI_{NoAb}” represents the average cell index value of the wells without antibody.

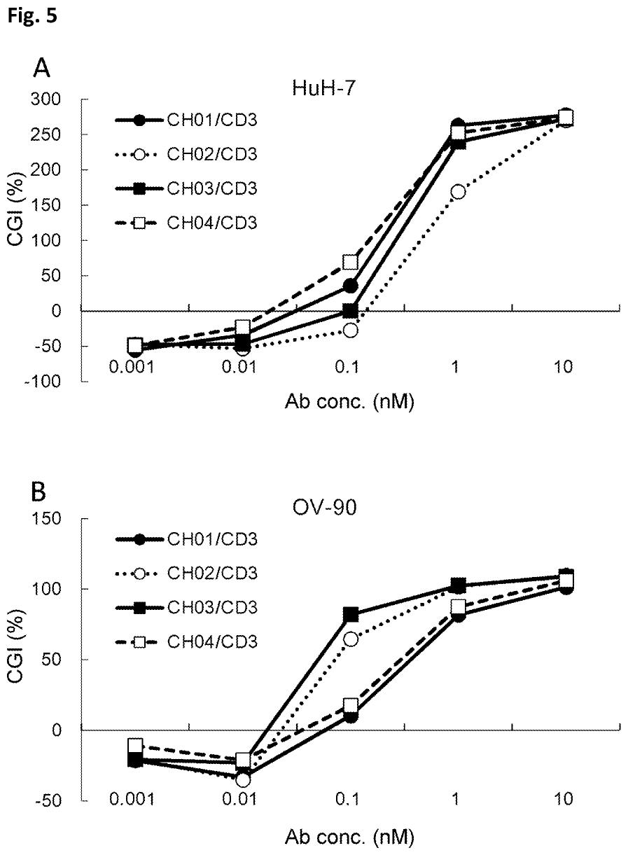

The results shows that all the CLDN6-TRABs inhibited the growth of CLDN6 expressing cancer cell lines in a dose dependent manner (FIG. 5).

### Example 6. In Vivo Anti-Tumor Efficacy Study

The in vivo anti-tumor efficacy of the CLDN6-TRABs was evaluated using tumor bearing mice model. The human cancer cell lines expressing human CLDN6 (HuH-7 or OV-90) was transplanted subcutaneously into NOD/ShiJic-scid mice, and human PBMC was injected into the mice (so-called T cell injection model). Tumor bearing mice were randomized to treatment groups to receive an administration of the antibody, or vehicle as a control, at day 14 from tumor inoculation.

More specifically, the following test was conducted in the efficacy test using HuH-7 or OV-90 T cell injection model. Expansion of T cells was performed using human PBMC (AllCells) and T Cell Activation/Expansion Kit (Miltenyi Biotec). 1×10⁷ cells of HuH-7 or 5×10⁶ cells of OV-90 were mixed with Matrigel Basement Membrane Matrix (BD), subjected to transplantation subcutaneously into the lateral region of the NOD/ShiJic-scid mice (CLEA Japan, Female, 6-weeks-old). The day of transplantation was defined as day 0. After randomization and grouping according to tumor size and body weight from day 14 after transplantation, anti-asialo GM1 antibody (Wako Pure Chemical Industries) was administered intraperitoneally at 0.2 mg/mouse. On the following day, the T cells obtained by the expansion culture were implanted by intraperitoneal injection at 3×10⁷ cells/mouse. Details of groups is shown in Table 4. Approximately four hours after T cell transplantation, CLDN6-TRABs (CH01/CD3, CH02/CD3, CH03/CD3, and CH04/CD3) were administered intravenously. CLDN6-TRABs was administered only once. The length (L) and width (W) of the tumor mass were measured, and tumor volume (TV) was calculated as: TV=(L×W×W)/2.

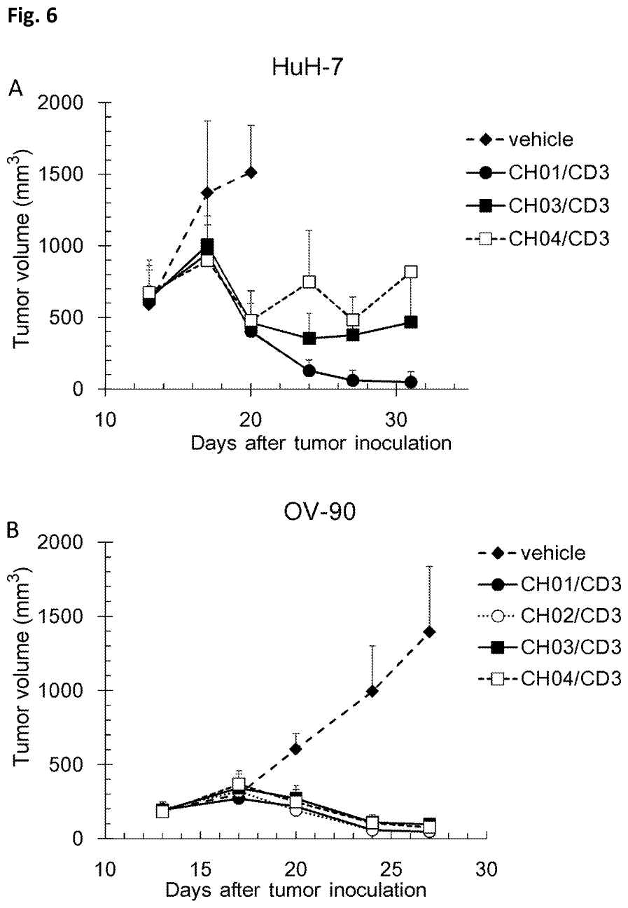

Anti-tumor efficacy was observed in the CLDN6-TRAB administering groups compared with the vehicle administering control group (FIG. 6). Especially, significant inhibition of tumor growth was observed in mice treated with CH01/CD3 in HuH-7 model.

TABLE 4
Details of study groups for in vivo anti-tumor efficacy evaluation

| | | |
|---|---|---|
| Group | Antibody | Dose |

a. Study groups using HuH-7 T cell injection model

| | | |
|---|---|---|
| 1 | vehicle | — |
| 2 | CH01/CD3 | 5 mg/kg |
| 3 | CH03/CD3 | 5 mg/kg |
| 4 | CB04/CD3 | 5 mg/kg |

b. Study groups using OV-90 T cell injection model

| | | |
|---|---|---|
| 1 | vehicle | — |
| 2 | CH01/CD3 | 5 mg/kg |
| 3 | CH02/CD3 | 5 mg/kg |
| 4 | CH03/CD3 | 5 mg/kg |
| 5 | CH04/CD3 | 5 mg/kg |

### Example 7. Pharmacokinetics of the CLDN6-TRABs in Mice

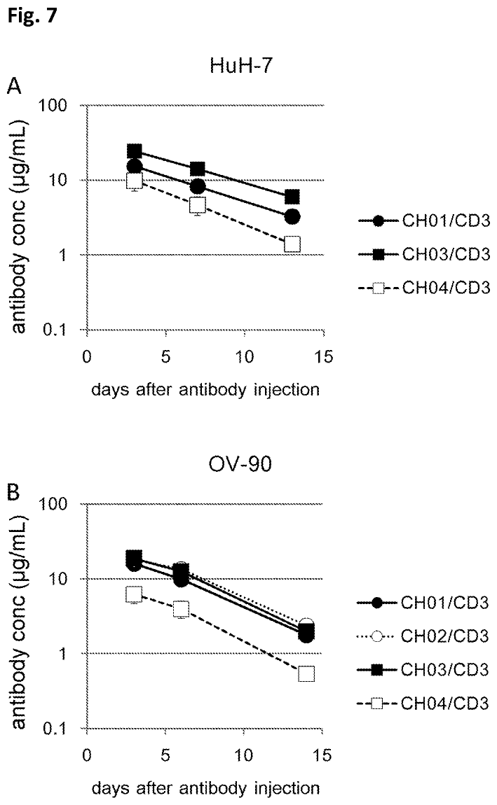

Pharmacokinetics of CLDN6-TRABs was evaluated as follows. The CLDN6-TRABs were injected intravenously into mice at a dose of 5 mg/kg. Blood was collected from three mice on day 3, 7, 13 after the antibody injection in HuH-7 transplant model and on day 3, 6, 14 after the antibody injection in OV-90 transplant model. The plasma samples in heparin was obtained by centrifuging at 10,000×g for 5 minutes at 4 degree C. The concentration of the CLDN6-TRABs in mouse plasma was measured by ELISA. Anti-human IgG antibody (Sigma) was dispensed onto a Nunc-ImmunoPlate MaxiSorp (Thermo Fisher) and kept for 1 hour at room temperature to prepare anti-human IgG immobilized plates. Samples having antibody concentrations at 8, 4, 2, 1, 0.5, 0.25 or 0.125 ng/mL for calibration, and mouse plasma samples diluted 400-fold or more were prepared. Each of the samples was dispensed into the anti-human IgG immobilized plates, and kept at room temperature for one hour. Then, biotinylated anti-Fc specific antibody (CHUGAI) was added to react for one hour at room temperature. Streptavidin-PolyHRP80 (Stereospecific Detection Technologies) was added to react for one hour at room temperature, and chromogenic reaction was carried out using ABTS (Roche) as a substrate. Then, the absorbance at 405 nm (650 nm as reference) was measured by a microplate reader. The antibody concentration in mouse plasma was calculated from the absorbance of the calibration curve using the analytical software SOFTmax PRO (Molecular Devices). The temporal change of CLDN6-TRAB (CH01/CD3, CH02/CD3, CH03/CD3 and CH04/CD3) concentration in mouse plasma after intravenous administration is shown in FIG. 7. The half-life (T v2) of the CLDN6-TRABs is approximately 3.44 to 4.46 days in the plasma of HuH-7 inoculated model and approximately 2.77 to 3.22 days in OV-90 inoculated model. Among the tested CLDN6-TRABs, CH04/CD3 showed the shortest half-life in both of the models. The area under the antibody concentration—time curve from zero to infinity (AUC_{inf}) of CH04/CD3 was the lowest both in HuH7 inoculated model and OV90 inoculated model.

TABLE 5
Antibody concentration of the CLDN6b-TRABs in mice plasma after infection

| | Parameters | t½ | AUClast | AUCinf | AUCext | CL | MRTlast | MRTinf | Vdss |
|---|---|---|---|---|---|---|---|---|---|
| Model | Unit | day | μg*day/mL | μday/mL | % | mL/day/kg | day | day | mL/kg |
| HuH7 | CH01/CD3 | 4.46 | 137 | 158 | 13.3 | 31.6 | 4.45 | 6.45 | 203 |
| | CH03/CD3 | 4.79 | 224 | 265 | 15.6 | 18.9 | 4.64 | 7.03 | 133 |
| | CH04/CD3 | 3.44 | 84.2 | 21.1 | 7.53 | 54.9 | 4.05 | 5.11 | 280 |
| OV90 | CH04/CD3 | 3.22 | 138 | 146 | 5.64 | 34.3 | 4.28 | 5.09 | 174 |
| | CH02/CD3 | 3.19 | 163 | 174 | 6.32 | 28.7 | 4.66 | 5.54 | 158 |
| | CH03/CD3 | 3.00 | 162 | 171 | 5.03 | 29.3 | 4.37 | 5.07 | 149 |
| | CH04/CD3 | 2.77 | 52.4 | 54.6 | 3.96 | 91.6 | 4.19 | 4.73 | 434 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### INDUSTRIAL APPLICABILITY

The present invention provides novel multispecific antigen-binding molecules that have a strong anti-tumor activity and an excellent safety property of not inducing a cytokine storm or such independently from cancer antigens, and have long half-lives in blood. Cytotoxicity-inducing agents that comprise an antigen-binding molecule of the present invention as an active ingredient can target CLDN6-expressing cells and tumor tissues containing these cells and induce cell injury. Administration of a multispecific antigen-binding molecule of the present invention to patients makes it possible to have a desirable treatment that has not only a high level of safety but also a reduced physical burden, and is highly convenient.

### SEQUENCE LISTING

1. A multispecific antigen-binding molecule that comprises:
(1) a first domain comprising a first antigen-binding domain which binds to human CLDN6,
(2) a second domain comprising a second antigen-binding domain which binds to T-cell receptor complex, and
(3) a third domain comprising an Fc region.

2. The multispecific antigen-binding molecule of claim 1, wherein the multispecific antigen-binding molecule has cytotoxic activity.

3. The multispecific antigen-binding molecule of claim 2, wherein the cytotoxic activity is T-cell-dependent cytotoxic activity.

4. The multispecific antigen-binding molecule of any one of claims 1 to 3, wherein the first antigen-binding domain of (1) specifically binds to human CLDN6.

5. The multispecific antigen-binding molecule of any one of claims 1 to 4, wherein the first antigen-binding domain of (1) does not substantially bind to at least one selected from human CLDN9, human CLDN4 and human CLDN3.

6. The multispecific antigen-binding molecule of any one of claims 1 to 5, wherein the first antigen-binding domain of (1) does not substantially bind to a CLDN6 mutant as defined in SEQ ID NO: 53.

7. The multispecific antigen-binding molecule of any one of claims 1 to 6, wherein the second antigen-binding domain in (2) binds to T-cell receptor.

8. The multispecific antigen-binding molecule of any one of claims 1 to 6, wherein the second antigen-binding domain in (2) binds to CD3 epsilon chain.

9. The multispecific antigen-binding molecule of any one of claims 1 to 8, wherein the first antigen-binding domain or the second antigen-binding domain is an antibody variable fragment, or both of the first and second antigen-binding domains are antibody variable fragments.

10. The multispecific antigen-binding molecule of claim 9, wherein the antibody variable fragment is a Fab.

11. The multispecific antigen-binding molecule of any one of claims 1 to 10, wherein the first antigen-binding domain of (1) is any one of (a1) to (a6) below:
(a1) an antibody variable region comprising the HVR-H1 sequence of SEQ ID NO: 1, the HVR-H2 sequence of SEQ ID NO: 2, the HVR-H3 sequence of SEQ ID NO: 3, the HVR-L1 sequence of SEQ ID NO: 4, the HVR-L2 sequence of SEQ ID NO: 5, and the HVR-L3 sequence of SEQ ID NO: 6;
(a2) an antibody variable region comprising the HVR-H1 sequence of SEQ ID NO: 9, the HVR-H2 sequence of SEQ ID NO: 10, the HVR-H3 sequence of SEQ ID NO: 11, the HVR-L1 sequence of SEQ ID NO: 12, the HVR-L2 sequence of SEQ ID NO: 13, and the HVR-L3 sequence of SEQ ID NO: 14;
(a3) an antibody variable region comprising the HVR-H1 sequence of SEQ ID NO: 17, the HVR-H2 sequence of SEQ ID NO: 18, the HVR-H3 sequence of SEQ ID NO: 19, the HVR-L1 sequence of SEQ ID NO: 20, the HVR-L2 sequence of SEQ ID NO: 21, and the HVR-L3 sequence of SEQ ID NO: 22;
(a4) an antibody variable region comprising the HVR-H1 sequence of SEQ ID NO: 25, the HVR-H2 sequence of SEQ ID NO: 26, the HVR-H3 sequence of SEQ ID NO: 27, the HVR-L1 sequence of SEQ ID NO: 28, the HVR-L2 sequence of SEQ ID NO: 29, and the HVR-L3 sequence of SEQ ID NO: 30;
(a5) an antibody variable region that binds to the same epitope with any one of the antibody variable regions selected from (a1) to (a4);
(a6) an antibody variable region that competes for the binding to human CLDN6 with any one of the antibody variable regions selected from (a1) to (a4).

12. The multispecific antigen-binding molecule of any one of claims 1 to 11, wherein the Fc region of (3) is an Fc region with reduced binding activity towards an Fc gamma receptor.

13. The multispecific antigen-binding molecule of claim 12, wherein the Fc region of is an Fc region with at least one amino acid mutation at any of the Fc region-constituting amino acids of SEQ ID NOs: 62 to 65 (IgG1 to IgG4).

14. The multispecific antigen-binding molecule of any one of claims 1 to 13, wherein the multispecific antigen-binding molecule is a bispecific antibody.

15. A pharmaceutical composition comprising the multispecific antigen-binding molecule of any one of claims 1 to 13, or the bispecific antibody of claim 14, and a pharmaceutically acceptable carrier.


SEQUENCE LISTING (84 sequences)

SEQ ID NO: 1 - protein, 5 aa, Artificial Sequence
  an artificial synthesized sequence
SYTMS

SEQ ID NO: 2 - protein, 17 aa, Artificial Sequence
  an artificial synthesized sequence
TISSGGGRTYYPDSVKG

SEQ ID NO: 3 - protein, 10 aa, Artificial Sequence
  an artificial synthesized sequence
GDYRYDGFAY

SEQ ID NO: 4 - protein, 11 aa, Artificial Sequence
  an artificial synthesized sequence
RASENIDSYLA

SEQ ID NO: 5 - protein, 7 aa, Artificial Sequence
  an artificial synthesized sequence
ASTLLVD

SEQ ID NO: 6 - protein, 9 aa, Artificial Sequence
  an artificial synthesized sequence
QHYYSIPYT

SEQ ID NO: 7 - protein, 119 aa, Artificial Sequence
  an artificial synthesized sequence
EVKLVESGGGLVKPGGSLKLSCAASGFTFNSYTMSWVRQTPAKRLEWVVTISSGGGRTYYPDSVKGRFTISRDNARNTLYLQMSSLRSEDTAMYYCIRGDYRYDGFAYWGQGTLVTVST

SEQ ID NO: 8 - protein, 107 aa, Artificial Sequence
  an artificial synthesized sequence
DIQMTQSPASLSASVGETVTITCRASENIDSYLAWYQQKQGKSPQLLVYASTLLVDGVPSRFSGSRSGTQFSLKINSLQSEDVARYYCQHYYSIPYTFGSGTKLEIK

SEQ ID NO: 9 - protein, 5 aa, Artificial Sequence
  an artificial synthesized sequence
EYTMH

SEQ ID NO: 10 - protein, 17 aa, Artificial Sequence
  an artificial synthesized sequence
GINPNNGNTRYNQKFKD

SEQ ID NO: 11 - protein, 12 aa, Artificial Sequence
  an artificial synthesized sequence
CGDYDLFFFFAY

SEQ ID NO: 12 - protein, 11 aa, Artificial Sequence
  an artificial synthesized sequence
KASQNVGTNVA

SEQ ID NO: 13 - protein, 7 aa, Artificial Sequence
  an artificial synthesized sequence
SASSRFS

SEQ ID NO: 14 - protein, 9 aa, Artificial Sequence
  an artificial synthesized sequence
QQYNSFPFT

SEQ ID NO: 15 - protein, 121 aa, Artificial Sequence
  an artificial synthesized sequence
EVQVEQSGLELVKPGASVKISCKTSGYTFTEYTMHWVKQSHGKSLEWIGGINPNNGNTRYNQKFKDKATLTVDKSSRTAYMELHTLTSEDSAVYYCARCGDYDLFFFFAYWGQGTLVTVSA

SEQ ID NO: 16 - protein, 107 aa, Artificial Sequence
  an artificial synthesized sequence
DIVMTQFQKFMSTSVGDRVSVTCKASQNVGTNVAWYQQKPGQSPEALISSASSRFSGVPDRFTGSGSGTDFTLTITNVQSEDLADYFCQQYNSFPFTFGSGTELEIK

SEQ ID NO: 17 - protein, 5 aa, Artificial Sequence
  an artificial synthesized sequence
EYTMH

SEQ ID NO: 18 - protein, 17 aa, Artificial Sequence
  an artificial synthesized sequence
GINPNNGNTRYNQKFKG

SEQ ID NO: 19 - protein, 12 aa, Artificial Sequence
  an artificial synthesized sequence
CGDFDLFFFFAY

SEQ ID NO: 20 - protein, 11 aa, Artificial Sequence
  an artificial synthesized sequence
KASQNVGSNVA

SEQ ID NO: 21 - protein, 7 aa, Artificial Sequence
  an artificial synthesized sequence
SASSRFS

SEQ ID NO: 22 - protein, 9 aa, Artificial Sequence
  an artificial synthesized sequence
QQYNSYPFT

SEQ ID NO: 23 - protein, 121 aa, Artificial Sequence
  an artificial synthesized sequence
EVQVEQSGPELVKPGASVKISCKTSGYTFTEYTMHWVKQSHGKSLEWIGGINPNNGNTRYNQKFKGKATLTVDKSSSTAYMELRTLTSEDSAVYYCARCGDFDLFFFFAYWGQGTLVTVSA

SEQ ID NO: 24 - protein, 107 aa, Artificial Sequence
  an artificial synthesized sequence
DIVMTQSQKFMSTSVGDRVSVTCKASQNVGSNVAWYQQKPGQSPEGLISSASSRFSGVPDRFTGSGSGTDFTLTITNVQSEDLAEYFCQQYNSYPFTFGSGTELEIK

SEQ ID NO: 25 - protein, 5 aa, Artificial Sequence
  an artificial synthesized sequence
GYTMN

SEQ ID NO: 26 - protein, 17 aa, Artificial Sequence
  an artificial synthesized sequence
LINPYNGGTIYNQKFKG

SEQ ID NO: 27 - protein, 8 aa, Artificial Sequence
  an artificial synthesized sequence
DYGFVLDY

SEQ ID NO: 28 - protein, 10 aa, Artificial Sequence
  an artificial synthesized sequence
SASSSVSYMH

SEQ ID NO: 29 - protein, 7 aa, Artificial Sequence
  an artificial synthesized sequence
STSNLAS

SEQ ID NO: 30 - protein, 10 aa, Artificial Sequence
  an artificial synthesized sequence
QQRSNYPPWT

SEQ ID NO: 31 - protein, 117 aa, Artificial Sequence
  an artificial synthesized sequence
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 32 - protein, 107 aa, Artificial Sequence
  an artificial synthesized sequence
DIVLTQSPSIMSVSPGEKVTITCSASSSVSYMHWFQQKPGTSPKLCIYSTSNLASGVPARFSGRGSGTSYSLTISRVAAEDAATYYCQQRSNYPPWTFGGGTKLEIK

SEQ ID NO: 33 - protein, 5 aa, Artificial Sequence
  an artificial synthesized sequence
NAWMH

SEQ ID NO: 34 - protein, 19 aa, Artificial Sequence
  an artificial synthesized sequence
QIKDKSQNYATYVAESVKG

SEQ ID NO: 35 - protein, 13 aa, Artificial Sequence
  an artificial synthesized sequence
RYVHYAAGYGVDI

SEQ ID NO: 36 - protein, 16 aa, Artificial Sequence
  an artificial synthesized sequence
RSSQPLVHSNRNTYLH

SEQ ID NO: 37 - protein, 7 aa, Artificial Sequence
  an artificial synthesized sequence
KVSNRFS

SEQ ID NO: 38 - protein, 9 aa, Artificial Sequence
  an artificial synthesized sequence
GQGTQVPYT

SEQ ID NO: 39 - protein, 122 aa, Artificial Sequence
  an artificial synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 40 - protein, 112 aa, Artificial Sequence
  an artificial synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 41 - protein, 328 aa, Artificial Sequence
  an artificial synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 42 - protein, 328 aa, Artificial Sequence
  an artificial synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 43 - protein, 107 aa, Artificial Sequence [sequence omitted]

SEQ ID NO: 44 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYV

SEQ ID NO: 45 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 46 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 47 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 48 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 49 - protein, 219 aa, Mus musculus
MASTGLQILGIVLTLLGWVNALVSCALPMWKVTAFIGNSIVVAQMVWEGLWMSCVVQSTGQMQCKVYDSLLALPQDLQAARALCVVTLLIVLLGLLVYLAGAKCTTCVEDRNSKSRLVLISGIIFVISGVLTLIPVCWTAHSIIQDFYNPLVADAQKRELGASLYLGWAASGLLLLGGGLLCCACSSGGTQGPRHYMACYSTSVPHSRGPPEYPTKNYV

SEQ ID NO: 50 - protein, 217 aa, Mus musculus
MASTGLELLGMTLAVLGWLGTLVSCALPLWKVTAFIGNSIVVAQVVWEGLWMSCVVQSTGQMQCKVYDSLLALPQDLQAARALCVVALLLALLGLLVAITGAQCTTCVEDEGAKARIVLTAGVLLLLSGILVLIPVCWTAHAIIQDFYNPLVAEALKRELGASLYLGWAAAALLMLGGGLLCCTCPPSHFERPRGPRLGYSIPSRSGASGLDKRDYV

SEQ ID NO: 51 - protein, 219 aa, Mus musculus
MSMGLEITGTSLAVLGWLCTIVCCALPMWRVSAFIGSSIITAQITWEGLWMNCVVQSTGQMQCKMYDSLLALPQDLQAARALIVVSILLAAFGLLVALVGAQCTNCVQDETAKAKITIVAGVLFLLAALLTLVPVSWSANTIIRDFYNPLVPEAQKREMGAGLYVGWAAAALQLLGGALLCCSCPPRDKYAPTKILYSAPRSTGPGTGTGTAYDRKDYV

SEQ ID NO: 52 - protein, 210 aa, Mus musculus
MASMGLQVLGISLAVLGWLGIILSCALPMWRVTAFIGSNIVTAQTSWEGLWMNCVVQSTGQMQCKMYDSMLALPQDLQAARALMVISIIVGALGMLLSVVGGKCTNCMEDETVKAKIMITAGAVFIVASMLIMVPVSWTAHNVIRDFYNPMVASGQKREMGASLYVGWAASGLLLLGGGLLCCSCPPRSNDKPYSAKYSAARSVPASNYV

SEQ ID NO: 53 - protein, 220 aa, Artificial Sequence
  an artificial synthesized sequence
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEALKRELGASLYLGWAASGLLLLGGGLLCCTCPSGGSQGPSHYMARYSTSAPAISRGPSEYPTKNYV

SEQ ID NO: 54 - protein, 141 aa, Homo sapiens
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 56 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 57 - protein, 204 aa, Homo sapiens
KQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKS

SEQ ID NO: 58 - protein, 177 aa, Homo sapiens
PSYTGGYADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 59 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 60 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 61 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 62 - protein, 330 aa, Homo sapiens [sequence omitted]

SEQ ID NO: 63 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 64 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 65 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 4 aa, Artificial Sequence
  an artificial synthesized sequence
GGGS

SEQ ID NO: 67 - protein, 4 aa, Artificial Sequence
  an artificial synthesized sequence
SGGG

SEQ ID NO: 68 - protein, 5 aa, Artificial Sequence
  an artificial synthesized sequence
GGGGS

SEQ ID NO: 69 - protein, 5 aa, Artificial Sequence
  an artificial synthesized sequence
SGGGG

SEQ ID NO: 70 - protein, 6 aa, Artificial Sequence
  an artificial synthesized sequence
GGGGGS

SEQ ID NO: 71 - protein, 6 aa, Artificial Sequence
  an artificial synthesized sequence
SGGGGG

SEQ ID NO: 72 - protein, 7 aa, Artificial Sequence
  an artificial synthesized sequence
GGGGGGS

SEQ ID NO: 73 - protein, 7 aa, Artificial Sequence
  an artificial synthesized sequence
SGGGGGG

SEQ ID NO: 74 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 75 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 76 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 77 - protein, 254 aa, Homo sapiens [sequence omitted]

SEQ ID NO: 78 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 79 - protein, 19 aa, Artificial Sequence
  an artificial synthesized sequence
MGWSCIILFLVATATGVHS

SEQ ID NO: 80 - DNA, 1125 bp, Homo sapiens
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 81 - DNA, 951 bp, Homo sapiens
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggggtcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 82 - DNA, 876 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 83 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 84 - DNA, 702 bp, Homo sapiens
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga